(19) United States
(12) Patent Application Publication
(10) Pub. No.: US 2004/0022777 A1
(43) Pub. Date: Feb. 5, 2004

(54) Fragments and antagonists of heat shock protein 60

(76) Inventors: Hubert Kolb, Duesseldorf (DE); Dana Elias, Gedera (IL)

Correspondence Address: WINSTON & STRAWN, PATENT DEPARTMENT, 1400 L STREET, N.W., WASHINGTON, DC, 20005-3502, US

(21) Appl. No.: 10/172,102

(22) Filed: Jun. 13, 2002

Related U.S. Application Data
(63) Continuation of application No. PCT/IL00/00833, filed on Dec. 14, 2000.
(60) Provisional application No. 60/172,297, filed on Dec. 15, 1999.

(30) Foreign Application Priority Data
Dec. 15, 1999 (EP) 00125035.8

(51) Int. Cl. C12N 9/99; A61K 38/43; C12P 21/02; C12N 5/06

(52) U.S. Cl. 424/94.1; 435/184; 435/69.2; 435/325; 435/320.1

(57) ABSTRACT

The invention identifies fragments of hsp60 which retain the capability of inducing a pro-inflammatory immune response of cells of the innate immune system. It is further discloses the unexpected finding that smaller peptides derived from these fragments of hsp60 are capable of acting as antagonists of hsp60 insofar as they have the ability to reduce or even prevent the induction of a pro-inflammatory immune response by hsp60 in cells of the innate immune system. It further discloses variants, derivatives and analogs of such peptides, which are capable of acting as antagonists of hsp60 insofar as they have the ability to reduce or prevent the induction of a pro-inflammatory immune response by hsp60 in cells of the immune system. The invention also discloses pharmaceutical compositions comprising such peptide fragments or variants, derivatives and analogs thereof, and their use to prevent or ameliorate inflammatory diseases or disorders.

### CROSS-REFERENCE TO RELATED APPLICATIONS

This application is a continuation of the US national stage designation of International application PCT/IL00/00833 filed Dec. 14, 2000, the content of which is expressly incorporated herein by reference thereto.

### FIELD OF THE INVENTION

The present invention relates to peptides and peptide analogs capable of acting as antagonists of hsp60 characterized in that they have the ability to reduce or prevent the induction of a pro-inflammatory response of the innate immune system by hsp60. The present invention further relates to pharmaceutical compositions comprising such peptides and peptide analogs or variants and derivatives thereof, useful for prevention or treatment of inflammatory disorders and autoimmune diseases.

### BACKGROUND OF THE INVENTION

Heat Shock Proteins

Heat shock proteins (hsp) are ubiquitous polypeptides produced by all cells of all species. Heat shock proteins are expressed both as constitutive proteins that act as molecular chaperones (Becker J. and Craig A. E., Eur. J. Biochem. 219;11, 1994), and as inducible stress proteins, and therefore are also referred to as stress proteins. The up-regulation of hsp expression within the cell was first described when cells were confronted with a sudden increase in temperature. Later, it was found that other insults could also induce hsp synthesis.

Molecular chaperones are proteins that play an essential role in post-translational assembly of oligomeric proteins in complexes, intracellular folding and transport of proteins, and “protection” of unfolded proteins from misfolding (denaturation) or unwanted protein-protein interactions.

Heat shock proteins are among the best conserved proteins phylogenetically, with respect to both sequence on the one hand and function on the other. Human and bacterial Heat shock proteins are more than 50% homologous (Jindal et al. Mol Cell Biol. 9:2279, 1989).

Hsp60 in Autoimmunity and Inflammation

Hsp60 is a mitochondrial chaperone with a major role in protein folding and unfolding as well as translocation of proteins into mitochondria. In the mitochondria it facilitates the refolding of these proteins, and if necessary their subsequent assembly into oligomeric complexes.

Hsp60 is found in the cell cytosol under stressful and inflammatory conditions; cell infection or elevated cytokine levels will induce the cellular stress response. Therefore, it is not surprising that hsp60 is a highly immunogenic protein: it is the “common antigen” of gram-negative bacteria. Immunological reactivity to both bacterial and autologous-hsp60 is highly prevalent in the general population, since the pathogen-directed immune response can easily convert into an autoimmune response due to the high homology.

T-cell responses to multiple hsp60 epitopes are present in various autoimmune and inflammatory diseases (van Eden et al. Immunology Today 19;303, 1998), including Type 1 diabetes (Elias et al. Proc Natl Acad Sci 88:3088, 1991), Rheumatoid and Juvenile arthritis, Multiple Sclerosis, Ankylosing Spondylitis, Pelvic Inflammation-associated infertility, Inflammatory Bowel Disease, Atherosclerosis, Graft rejection and more. The immune system reacts to hsp60 epitopes that are either cross-reactive between the human and bacterial analogues, or idiosyncratic.

Immunological tolerance has never been achieved so far to hsp60 epitopes in experimental systems, indicating that the immune system is predisposed to recognize this particular protein. It is a strongly immunogenic protein, in the general (healthy) population as well as in patients with inflammatory disorders.

Continuous exposure to benign and pathogenic bacteria acts as a booster for hsp60 responses, by re-exposing the host to bacterial hsp60. Moreover, inflammatory cytokines that are secreted during infection elevate local tissue expression of self-hsp60. Cells damaged during inflammation release high amounts of self-hsp60 to an immunologically active environment, leading to immunity.

Inflammatory diseases associated with hsp60 expression in target tissues include:

(i) Autoimmune diseases: Diabetes (Birk et al. Proc Natl Acad Sci 93:1032, 1996), Multiple Sclerosis, Rheumatoid arthritis (van Eden et al., Nature, 331:171, 1988), juvenile chronic arthritis

(ii) Chronic inflammation: Inflammatory Bowel Disease, Reactive Arthritis.

(iii) Graft rejection (Birk et al. Proc Natl Acad Sci, 96:5159, 1999).

(iv) Atherosclerosis.

Hsp60 and Atherosclerosis:

Hsp60 has been implicated in atherosclerosis, since autoantibodies to human hsp60 were demonstrated to correlate with the clinical status of patients and experimental animal models. The suggested mechanism was that autoreactive hsp60-specific T-cells respond to hsp60 that is over-expressed on endothelial cells of the aortic intima. An alternative mechanism is suggested by the finding that hsp60 is expressed in 89% of macrophages in the atheroma. Moreover, hsp60 can stimulate macrophage functions relevant to atherosclerosis, such as the production of TNFα, IL-6 and matrix-degrading metalloproteinases. Hsp60 acts on the endothelial cells as well, inducing the production of E-selectin, ICAM-1, VCAM-1 and IL-6. The adhesion molecules enhance the trafficking of monocytes and lymphocytes to the atheroma, while the pro-inflammatory cytokines will enhance plaque instability and thrombosis. Therefore, hsp60 can activate the expression of an array of molecules that promote atherogenesis in both the target and effector cells.

Use of Heat Shock Proteins in Therapy

Several disclosures claim uses of heat shock proteins as immune modulators in diagnosis, treatment or prevention of autoimmune diseases. Most of the patent documents relate to bacterial heat shock protein 60 also known previously as hsp65, or fragments of these proteins.

For example, the particular protein produced by the human body during development of IDDM, which serves as a diagnostic marker for the incipient outbreak of IDDM, is the human heat shock protein having a size of about 65 kD (human hsp65) or an antigen cross-reactive therewith as disclosed in European patent 0417271, and in U.S. Pat. Nos. 5,114,844; 5,671,848; 5,578,303 and 5,780,034.

It has been disclosed that fragments of this hsp60 protein may serve as therapeutically useful entities in preventing or alleviating IDDM and host vs. graft disease (International Patent applications WO 96/19236, WO 97/01959 and WO 98/08536).

In addition, fragments of hsp60 may be used as carriers for development of synthetic vaccines by increasing the immunogenicity of poorly immunogenic antigens as disclosed in U.S. Pat. Nos. 5,736,146 and 5,869,058.

European patent 0262710 discloses polypeptides useful for alleviation, treatment, and diagnosis of autoimmune arthritis and similar autoimmune diseases. The claimed polypeptides are derived from bacterial protein named “Antigen A” which was later identified as mycobacterial hsp60.

PCT application WO 92/04049 discloses peptides of at least seven amino acids homologous to a fragment of Mycobacterium tuberculosis hsp60, which inhibit T-lymphocytes activation and proliferation and can protect from immune reactions and immune-related disease.

PCT applications WO 89/12455 and WO 94/29459, disclose the use of stress proteins and analogs for producing or enhancing an immune response or for inducing immune tolerance, for prophylaxis or therapy of autoimmune diseases and for treating or preventing infectious or cancers. A fusion protein is claimed comprising a stress protein fused to a protein against which an immune response is desired.

PCT application WO 95/25744 discloses microbial stress protein fragments containing epitopes homologous to related mammalian epitopes—used to treat and prevent inflammatory autoimmune diseases and to prevent transplant rejection. The protective epitopes are located in short 5-15 AAs regions of stress proteins, which are highly conserved between microorganisms and animals.

PCT applications WO 97/11966 and WO 96/10039 disclose polypeptides of up to 21 amino acids, derived from microbial heat shock protein which are useful for prophylaxis or treatment of autoimmune diseases especially arthritis.

PCT application WO 96/16083 discloses a peptide 25 amino acids long, derived from the 10 kD heat shock protein (hsp10) of Mycobacterium tuberculosis which is useful in pharmaceutical products for the treatment of inflammatory pathologies, especially rheumatoid arthritis.

PCT application WO 91/02542 discloses the use of antigenic and/or immuno-regulatory material derived from Mycobacterium vaccae and specifically hsp60, for treating chronic inflammatory disorders caused or accompanied by an abnormally high release of IL-6 and/or TNFα.

PCT application WO 96/18646 discloses peptides of 9-20 amino acids derived from Mycobacterial hsp60 used for treatment or prevention of autoimmune CNS diseases, e.g. multiple sclerosis, chronic inflammatory CNS diseases and primary brain tumors.

Other inventors use heat shock proteins other than hsp60 for treatment. For example, U.S. Pat. No. 5,348,945 discloses a method for reducing mortality in stressed tissue with heat shock protein for treatment of atherosclerosis, arterial restenosis and anoxic nerve damage using exogenous hsp70.

Other inventions (e.g. JP 10212230, JP 09241159) disclose synthetic and natural compounds and extracts which inhibit the expression of proteins belonging to the hsp60 or hsp27 families and are therefore useful for treating autoimmune diseases and cancers.

Toll Proteins

International application WO 99/20756 discloses DNA sequences encoding the human Toll proteins and antibodies specifically binding these proteins. The polypeptides are used to identify other proteins involved in Toll-mediated transduction (e.g. natural ligands), to screen for receptor and ligand mimics, and to generate antibodies.

International application WO 98/50547 discloses human DNA of Toll-like receptors and proteins and peptides derived from them. The compounds are claimed for use in altering phosphate metabolism, modulating inflammatory function or innate immunity responses. A binding compound, preferably an antibody or antibody fragment which specifically binds to these proteins or peptides, is also disclosed.

However, the exact role of the Toll proteins in the above-mentioned processes has not been elucidated.

Thus, there is a need to modulate the role of autologous hsp60 and possibly of Toll-like receptor complexes in influencing certain diseases and in particular in the regulation of pro-inflammatory immune responses and to provide molecules which are useful to suppress or even prevent such responses.

None of the background art teaches or suggests the hsp60 peptides, variants, derivatives and analogs disclosed herein which are useful as antagonists of hsp60 and have the ability to reduce or prevent the induction of a pro-inflammatory immune response by hsp60.

### SUMMARY OF THE INVENTION

The present invention provides novel anti-inflammatory peptides of hsp60, which are capable of acting as antagonists of hsp60 characterized in that they have the ability to reduce or prevent the induction of a pro-inflammatory immune response of cells of the innate immune system by hsp60. The present invention also provides variants, derivatives and analogs of peptides of hsp60, which are also capable of acting as antagonists of hsp60 characterized in that they have the ability to reduce or prevent the induction of a pro-inflammatory innate immune response by hsp60. The antagonists according to the present invention will preferably be peptides comprising 5 to 30 amino acid residues, more preferably 5 to 25 amino acid residues, most preferably 7 to 18 amino acid residues, as well as their variants, derivatives or analogs.

The present invention has identified those parts of hsp60 that are relevant for eliciting a pro-inflammatory immune response and to provide molecules that reduce or prevent such a response. Thus, the present invention provides fragments of the human hsp60 protein having a molecular weight of about 20 kD or less and which retain the ability of hsp60 to induce a pro-inflammatory innate immune response. These fragments are particularly useful according to the present invention, for the design of variants, derivatives and analogs which can serve as antagonists of the inflammatory response induced by hsp60.

According to the principles of the present invention, certain pro-inflammatory fragments of hsp60 were identified in order to serve as useful leads in the design of inhibitory variants, derivatives and analogs. Unexpectedly, it is now disclosed that shorter peptides from within these fragments possess anti-inflammatory activity characterized in that they can reduce or even prevent the pro-inflammatory responses induced by hsp60. These shorter peptides will be referred to herein as anti-inflammatory peptides of hsp60.

In general the anti-inflammatory peptides, derivatives and variants of the present invention may be derived from epitopes of hsp60 that undergo processing and are not necessarily exposed in the native protein. Conversely, they may be derived from epitopes or loops of the protein which are exposed on the surface of the hsp60 molecule.

According to the present invention it is now disclosed that the currently more preferred analogs are designed to mimic certain epitopes of the molecule designated hsp60. Most preferred peptides include analogs of the human hsp60 while additional preferred analogs include analogs of bacterial hsp60.

According to the most preferred embodiment of the present invention, the anti-inflammatory peptides, as well as the variants, derivatives and analogs of the fragments disclosed and claimed, are characterized by their ability to act as antagonists of the inflammatory responses induced by hsp60.

Another embodiment of the present invention provides cyclic derivatives and analogs of heat shock proteins or peptides derived therefrom having improved properties compared to the linear sequence to which they correspond. These properties include but are not limited to prolonged metabolic stability or enhanced selectivity of action.

In a preferred embodiment of the invention, these variants and derivatives of the fragments are further modified to backbone-cyclized peptide analogs that incorporate at least one building unit containing one nitrogen atom of the peptide backbone linked to a bridging group. Preferably these cyclic peptide analogs will correspond to 5 to 30 amino acids residues of a linear peptide sequence. More preferred backbone cyclized analogs have 5-18 residues with at least one building unit connected to a terminal, a side chain or another building unit to form a cyclic peptide analog. In particular, it is generally desirable to achieve peptide analogs with greater specificity thereby achieving enhanced clinical selectivity.

The currently most preferred anti-inflammatory peptides of the invention and variants, derivatives or analogs of the peptide sequences are now disclosed:

Preferred embodiments are derived from the sequence of the human hsp60. Additional preferred embodiments are derived from the sequence of the bacterial hsp60.

Preferred embodiments according to the present invention include anti-inflammatory peptides, variants, derivatives and analogs of fragments having a sequence selected from the group consisting of:

a. Residues 126-160 of human hsp60. A more preferred portion of this region spans residues 126-156 of human hsp60.

b. Residues 140-192 of human hsp60. More preferred portions of this region comprise analog spanning residues 157-191, 158-192, 158-191, 161-191, and 161-192 of human hsp60.

c. Residues 220-250 of human hsp60. A most preferred portion of this region spans residues 222-249 of human hsp60.

d. Residues 269-290 of human hsp60.

e. Residues 310-344 of human hsp60. Preferred portion of this region spans residues 315-344 of human hsp60.

f. Residues 390-421 of human hsp60.

g. A currently most preferred embodiment according to the present invention comprises derivatives, variants and peptide analogs corresponding to portions of residues 350-400 of the human hsp60.

Additional most preferred peptides and peptide analogs according to the present invention are:

i. Tyr-Phe-Ile-Asn-Thr-Ser-Lys-Gly-Gln-Lys corresponding to residues 227-236 of human hsp60

ii. Lys-Ala-Gln-Ile-Glu-Lys-Arg-Ile-Gln-Glu corresponding to residues 364-373 of human hsp60

iii. Lys-Arg-Ile-Gln-Glu-Ile-Ile-Glu-Gln-Leu corresponding to residues 369-378 of human hsp60

iv. Ile-Gln-Glu-Ile-Ile-Glu-Gln-Leu-Asp-Val corresponding to residues 371-380 of human hsp60

v. Gln-Glu-Ile-Ile-Glu-Gln-Leu-Asp-Val-Thr corresponding to residues 372-381 of human hsp60

vi. Val-Leu-Lys-Val-Gly-Gly-Thr-Ser-Asp-Val corresponding to residues 403-412 of human hsp60

vii. Lys-Val-Gly-Gly-Thr-Ser-Asp-Val-Glu-Val corresponding to residues 405-414 of human hsp60

Additional more preferred derivatives, variants and peptides analogs according to the present invention are based on residues 364-381 of the human hsp60 sequence: Lys-Ala-Gln-Ile-Glu-Lys-Arg-Ile-Gln-Glu-Ile-Ile-Glu-Gln-Leu-Asp-Val-Thr. The preferred anti-inflammatory peptides and peptide analogs of this sequence comprise 10-18 amino acid residues with optionally at least one residue replaced with an Alanine. The currently most preferred peptide analogs are:

Lys-Ala-Gln-Ile-Glu-Lys-Arg-Ile-Gln-Glu-Ile-Ile-Ala-Gln-Leu corresponding to residues 367-381 of human hsp60;

Lys-Arg-Ala-Gln-Glu-Ile-Ile-Glu-Ala-Leu corresponding to residues 372-381 of human hsp60.

Another preferred embodiment according to the present invention comprises derivatives, variants and peptide analogs of residues 403-412 of the human hsp60. The most preferred analogs of this region include the following sequences:

Val-Leu-Lys-Val-Gly-Gly-Thr-Ser-Asp-Val-Glu-Val-Asn;

Ala-Val-Leu-Lys-Val-Gly-Gly-Thr-Ser-Asp-Val-Glu-Val;

Val-Ala-Val-Leu-Lys-Val-Gly-Gly-Thr-Ser-Asp-Val-Val;

Gly-Val-Ala-Val-Leu-Lys-Val-Gly-Gly-Thr-Ser-Asp-Val.

Yet other more preferred analogs according to the present invention are backbone cyclized peptide derivatives, variants and analogs of residues 408-416 of the human hsp60. These analogs optionally have a Gln, Ser, Thr, Ala, Leu or 2Abu residue at position 415 instead of Asn.

Currently most preferred analogs of this sequence include backbone cyclized nonapeptides comprising two bridging groups at positions selected from the group consisting of: residues 408 and 414, residues 409-416, residues 408-416, and residues 409-414. Other preferred backbone cyclized analogs have 5-18 residues with at least one building unit connected to a terminal, a side chain or another building unit to form a cyclic peptide analog.

All the sequences listed above and in the examples are useful per se and may also be used as a basis for the design and synthesis of derivatives, variants and analogs with improved properties.

The invention also includes pharmaceutical compositions comprising a variant, derivative or analog of a fragment that can act as an antagonist of hsp60, and, optionally a pharmaceutically acceptable carrier or diluent.

The present invention furthermore relates to a method for the preparation of a pharmaceutical composition comprising a variant or derivative of a fragment as described herein. The formulation of the compound into a pharmaceutical composition might further comprise the addition of a pharmaceutically acceptable carrier, excipient and/or diluent.

The pharmaceutical compositions comprising pharmacologically active analogs of heat shock proteins and a pharmaceutically acceptable carrier or diluent represent another embodiment of the invention, as do the methods for the treatment of chronic inflammatory diseases, graft rejection and autoimmune diseases using such compositions.

The pharmaceutical compositions according to the present invention advantageously comprise at least one anti-inflammatory peptide or peptide analog, the sequence of which is derived from or mimics a portion of a heat shock protein molecule. These pharmaceutical compositions may be administered by any suitable route of administration, including orally, topically or systemically. Preferred modes of administration include but are not limited to parenteral routes such as intravenous and intramuscular injections. Additional preferred routes of administration include but are not limited to administration via nasal inhalation or oral ingestion.

The present invention also provides methods for identifying, screening and/or characterizing compounds which can act as antagonists of hsp60 insofar as they can reduce or prevent the induction of a pro-inflammatory response by hsp60. The compound to be tested can be any possible compound, e.g. variants and derivatives of fragments of hsp60 as described above, inorganic or organic compounds, antibodies, e.g. monoclonal or polyclonal antibodies, antibody fragments, such as Fab fragments, soluble Toll-like receptor 4 or Toll-like receptor 2 or fragments or complexes of these receptors, etc.

The methods for identifying, screening and/or characterizing fragments of hsp60 having the ability to elicit a pro-inflammatory immune response and/or for identifying compounds which can act as antagonists of hsp60 according to the present invention may also comprise high-throughput and ultra high-throughput quantitative and/or qualitative screening methods.

The compounds of the present invention are useful as active ingredients in pharmaceutical compositions for the prevention or treatment of diseases involving heat shock proteins in their etiology or pathology.

In this respect, the present invention provides molecules that are useful in suppression or prevention of certain diseases and conditions in which hsp60 is responsible for induction of a pro-inflammatory immune response of cells of the innate immune system.

The present invention relates therefore to the use of a variant, derivative or analog of a fragment of hsp60 according to the invention for the preparation of a pharmaceutical composition for the treatment of an inflammatory disease, such as an autoimmune disease (including but not limited to diabetes, multiple sclerosis, rheumatoid arthritis, myasthenia gravis and systemic lupus erythematosis), chronic inflammation (including but not limited to atherosclerosis, inflammatory bowel disease, reactive arthritis), chronic infections, graft rejection, gingivitis, gastritis, ulcer, thrombosis, allergy and acute infections.

Disclosures in the background art relate to numerous specific peptides derived from hsp60, which are useful in the treatment of specific diseases or disorders based on the interaction of those peptides with specific T-cell receptors. The present invention is directed to novel peptides of hsp60 which are useful for the suppression or prevention of innate immune responses mediated by other cells of the immune system, and excludes all of the previously known hsp60 peptides having the capacity to modulate T cell responses.

### BRIEF DESCRIPTION OF THE DRAWINGS

The invention will better be understood in relation to the drawings and detailed description of the preferred embodiments which follow:

FIG. 1 shows differential effects of human hsp60 on TNFα production from bone marrow-derived macrophages with wild-type or mutant Tlr4.

FIG. 2 shows dose dependency and time course of hsp60 induced TNFα production.

FIG. 3 shows differential effects of human hsp60 on NO production from bone marrow-derived macrophages with wild-type or mutant Tlr4.

FIG. 4 shows dose dependency and time course of the hsp60 induced NO production.

FIG. 5 shows the elution profile of the gel filtration chromatography of tryptic fragments of hsp60 on a Superdex-75 FPLC column (described in Example 2, section 1).

FIG. 6 shows the results of a reverse phase HPLC on a C-4 column of a subfraction of the tryptic fragments obtained from hsp60 (described in Example 2, section 2).

FIG. 7 shows a mass spectral analysis of peak no. 3 in the HPLC separation (FIG. 6) which shows a molecular weight of 3441 Daltons (corresponding to Glu₁₂₆ to Lys₁₅₆ or to Leu₃₉₀ to Arg420).

FIG. 8 shows the results of the testing of hsp60 and tryptic fragments of hsp60 for their ability to induce the production of TNFα in macrophages (Example 1, section 1).

FIG. 9 shows the results of the testing of hsp60 and tryptic fragments of hsp60 for their ability to induce the production of NO in macrophages (Example 1, section 1).

FIG. 10 describes the results of screening of the decapeptides for inhibition of hsp60 (Example 7).

FIG. 11 describes schematically the design of 96 peptide analogs based on the region of residues 364-381 of human hsp60 (Example 8).

### DETAILED DESCRIPTION OF THE PREFERRED EMODIMENTS

The present invention relates to identification of fragments of hsp60 that are capable of inducing a pro-inflammatory immune response by cells of the innate immune system. More particularly the present invention relates to even smaller anti-inflammatory peptides, variants and derivatives of these fragments which are capable of acting as antagonists of hsp60 insofar as they have the ability to reduce or prevent the induction of a pro-inflammatory immune response by hsp60. The present invention also relates to pharmaceutical compositions comprising such peptides or variants and derivatives thereof.

The present invention is based on the finding that hsp60 binds to the Toll-like receptor 4 complex and as a result elicits a potent pro-inflammatory response in cells of the innate immune system as exemplified hereinbelow. It was furthermore found that such a response can also be elicited by smaller fragments of hsp60 as described above. With the knowledge provided by the present invention it is now possible and to identify those regions of hsp60 which are crucial for eliciting a pro-inflammatory response in cells of the innate immune system.

Unexpectedly, it is now disclosed that it is possible to identify even smaller peptides of hsp60 which possess anti-inflammatory properties, and that these peptides are able to reduce or prevent the inflammatory response elicited by exposure to intact hsp60.

One approach is to build a 3-dimensional model of hsp60 and to determine which of the above mentioned amino acid stretches of (a) to (l) are expected to be exposed to the solvent. A 3-dimensional model of hsp60 has been built by homology from the crystallographically known structure of its bacterial homolog GROEL Protein DataBase entry PDB 1grl.ent (see also Braig et al., Nature 371 (1994), 578). Thus, it is possible to, e.g., chemically synthesize those amino acid stretches which were identified as being exposed to the solvent as well as smaller fragments thereof and to test them for their ability to elicit either a pro-inflammatory or an anti-inflammatory response using the assays as described above.

Furthermore, based on the knowledge that hsp60 binds to the Toll-like receptor 4 complex, it is now possible to compare the sequence of hsp60 with that of the spaetzle protein, which is the natural ligand of the Toll receptor in Drosophila where Toll controls dorso-ventral patterning (Marisato and Anderson, Cell 76 (1994), 677). By this comparison (see scheme I) 6 regions of homology can be determined, allowing thereby the identification of those amino acid sequences within hsp60 which can potentially bind to the recognition sites of the Toll-like receptor 4 complex.

It has unexpectedly been discovered that smaller hsp6O peptides, identified within these epitopes or fragments of hsp60, can themselves exert anti-inflammatory activity, inasmuch as they reduce or prevent the response to hsp60, as exemplified extensively hereinbelow.

Moreover, it is possible to predict the secondary structure of the spaetzle protein. This allows identification of fragments of hsp60 which show a structural similarity to corresponding parts of the spaetzle protein. Analysis of the six regions showed:

Scheme I. (legend: * identical residues, :very similar residues, . similar residues).

Sequence Alignment Between spaetzle and hsp60

| | |
|---|---|
| spaetzle hsp60 | 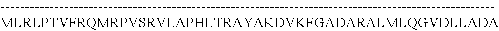 |
| spaetzle hsp60 | 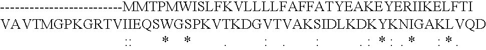 |
| spaetzle hsp60 | 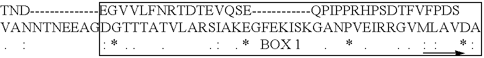 |
| spaetzle hsp60 | 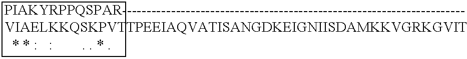 |
| spaetzle hsp60 | 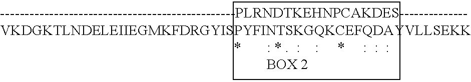 |
| spaetzle hsp60 | 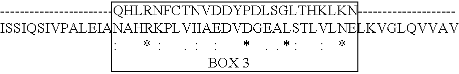 |
| spaetzle hsp60 | 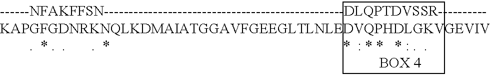 |
| spaetzle hsp60 | 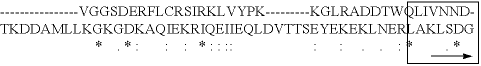 |
| spaetzle hsp60 | 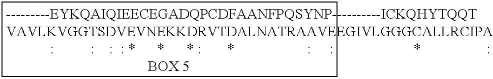 |
| spaetzle hsp60 | 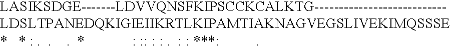 |
| spaetzle hsp60 | 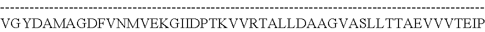 |
| spaetzle hsp60 | 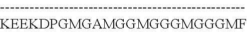 |

Region 1 shows a significant degree of homology with two fragments of spaetzle (this region and the alignment are marked as Box 1 in Scheme I. The arrow in the box denotes that the fragment continues on the next line).

Region 2 does not show sequence homology to spaetzle, yet spans two solvent exposed helices within hsp60, along with two loops, one preceding the first helix and the other between the two. Both the loops and the helices are solvent exposed and are thus potential recognition sites on hsp60. Since spaetzle is predicted to be a four-helix bundle, a structural homology between Region 2 and spaetzle that enables recognition by the same receptor is possible, despite lack of obvious sequence homology within this region.

Region 3 shows significant homology with a single fragment of spaetzle (marked as Box 2 in Scheme I). This region is a highly flexible, surface exposed loop in the GROEL crystal structure, and hence in the hsp60 model. The secondary structure of spaetzle in the region homologous to Region 3 is also predicted to be a loop. Thus, all of the analyses suggest this fragment to be a likely candidate for recognition by the toll receptor.

Region 4 shows significant homology with a fragment of spaetzle (marked as Box 3 in Scheme I). It is only partially exposed in the hsp60 model. Structural comparison between this region and the corresponding region in spaetzle is not straightforward, since a large part of this fragment shows poor electron density in the GROEL crystal.

Region 5 spans a large fragment of hsp60 that shows no sequence homology with spaetzle tailed by a short fragment that shows homology (Box 4 in Scheme I). The C-terminal part of this fragment (which includes the Box 4 sequence and an additional 9 residues upstream, are surface exposed).

Region 6 shows homology with two fragments of spaetzle (Box 5 in Scheme I; the arrow marks the continuation of this region to the next line). The entire fragment consists of a surface exposed helix, followed by a short β-strand, a short, partially exposed loop and an additional helix. The spaetzle fragment homologous to the first helix is predicted to be a loop, yet the fragment homologous to the C-terminal helix is predicted to be a helix also.

An additional region of hsp60 spanning Lys359 to Asp399 shows a significant degree of homology to a fragment of spaetzle, and is solvent exposed.

In one currently preferred embodiment of the present invention the fragment of the human hsp60 protein is obtainable by tryptic digest of human hsp60, in particular by treating an hsp60 protein with trypsin preferably at an enzyme/protein weight ratio of 1:54, incubating the reaction mixture at 37° C. for 0.5, 1.5, 5.4 and 16.5 hours, respectively, combining these reactions mixtures after having stopped the reaction, centrifuging them, filtering the supernatant and submitting it to a gel filtration chromatography on a Superdex-75 FPLC column with PBS in a rate of 0.5 ml/min, collecting fractions, optionally concentrating them and assaying them for their ability to induce a pro-inflammatory response as described above. For further purification a fraction which is active in the test for a pro-inflammatory response can be subjected to a reverse phase HPLC on a C-4 column using a gradient of 0.1% TFA in water and 0.1% TFA in acetonitryl, collecting fractions and again testing them for their ability to induce a pro-inflammatory immune response.

In a further more preferred embodiment the fragment according to the invention contains at least one of the following amino acid stretches present in the native hsp60 protein:

(a) Glu₁₂₆ to Lys₁₅₆;

(b) Lys₁₅₇to Lys₁₉₁;

(c) Gln₁₅₈ to Lys₁₉₂;

(d) Gln₁₅₈ to Lys₁₉i;

(e) Pro₁₆₁ to Lys₁₉₁;

(f) Pro₁₆₁ to Lys₁₉₂;

(g) Gly₂₂₂ to Lys₂₄₉;

(h) Lys₂₆₉ to Arg₂₉₀;

(i) Lys₃₁₀ to Lys₃₄₄;

(j) Asp₃₁₅ to Lys₃₄₄;

(k) Leu₃₉₀ to Val₄₂₁; and

(l) Thr₅₂₇ to Gly₅₇₁,

wherein the numbers given as indices represent the position of the corresponding amino acid residue of the human hsp60 protein as disclosed in SWISSPROT database accession No. P10809.

The hsp60 fragments of the present invention, as well as their derivatives and/or variants may be linked to other (poly)peptide sequences or may be part of (a) fusion protein(s). Such (poly)peptides/fusion proteins may be engineered to improve the characteristics of the fragments, derivatives or variants. For example, further amino acids may be added to improve stability and/or persistence during purification, handling or storage processes or to improve stability, half-life and/or persistence in host organisms and/or patients.

Furthermore, the hsp60 fragments, derivatives and/or variants of the invention may be fused to other proteins or peptides which play a role in inflammatory immune responses or in their potential treatment. Within the scope of the present invention are also molecules which comprise the hsp60 fragments, derivatives or variants of the invention which are linked to marker molecules and/or marker amino acid sequences. Such sequences comprise but are not limited to peptide-tags, histidine-tags, fluorescent molecules, GFP, FLAG and GST.

In a preferred embodiment of the variants and derivatives of the fragments according to the invention these molecules are further modified so as to be “backbone cyclized peptide analogs”. Backbone cyclized peptide analogs comprise a peptide sequence of preferably 5 to 30 amino acids that incorporates at least one building unit containing one nitrogen atom of the peptide backbone. In particular, it is generally desirable to achieve peptide analogs with greater specificity thereby achieving enhanced clinical selectivity. As a result of major advances in organic chemistry and in molecular biology, many bioactive peptides can now be prepared in quantities sufficient for pharmacological and clinical utilities. Thus in the last few years new methods have been established for the treatment and therapy of illnesses in which peptides have been implicated. However, the use of peptides as drugs is limited by the following factors: a) their low metabolic stability towards proteolysis in the gastrointestinal tract and in serum; b) their poor absorption after oral ingestion, in particular due to their relatively high molecular mass or the lack of specific transport systems or both; c) their rapid excretion through the liver and kidneys; and d) their undesired side effects in non-target organ systems, since peptide receptors can be widely distributed in an organism.

Thus, it would be most beneficial to produce conformationally constrained peptide analogs overcoming the drawbacks of the native peptide molecules, thereby providing improved therapeutic properties. One conceptual approach to the conformational constraint of peptides was introduced by Gilon et al. (Bio-polymers 31 (1991), 745) who proposed backbone to backbone cyclization of peptides. The theoretical advantages of this strategy include the ability to effect cyclization via the carbons or nitrogens of the peptide backbone without interfering with side chains that may be crucial for interaction with the specific receptor of a given peptide. While the concept was envisaged as being applicable to any linear peptide of interest, in point of fact the limiting factor in the proposed scheme was the availability of suitable building units that must be used to replace the amino acids that are to be linked via bridging groups. The actual reduction to practice of this concept of backbone cyclization initially was limited by the inability to devise any practical method of preparing building units of amino acids other than glycine (Gilon et al., J. Org. Chem. 587 (1992), 5687). Further disclosures by Gilon and coworkers (WO 95/33765, WO 97/09344, U.S. Pat. Nos. 5,723,575, US 5,811,392 and US 5,883,293) provided methods for producing building units required in the synthesis of backbone cyclized peptide analogs. Recently, the successful use of these methods to produce backbone cyclized peptide analogs of bradykinin analogs (U.S. Pat. No. 5,874,529), and backbone cyclized peptide analogs having somatostatin activity was also disclosed (WO 98/04583 and U.S. Pat. No. 5,770,687). All of these methods are incorporated herein in their entirety, by reference.

The present invention also provides a method for identifying fragments of hsp60 having the ability to elicit a pro-inflammatory immune response comprising the steps of

contacting cells carrying on their surface an intact Toll-like receptor complex with a fragment of hsp60; and

determining the amount of a pro-inflammatory mediator or effector, produced by the cells;

wherein an increase in the production of the pro-inflammatory mediator or effector, is indicative for the ability of the fragment of hsp60 to elicit a pro-inflammatory response. This method is exemplified herein for the pro-inflammatory mediators TNFα and/or NO.

The fragment of hsp60 which is used in step (a) can be any possible fragment of hsp60. Preferably, such a fragment has a length of at least 5 amino acid residues, more preferably of at least 10, even more preferably of at least 20 and most preferably of at least 50 amino acid residues. Such a fragment may be produced, e.g., by cleavage of the hsp60 protein, for example by proteolytic cleavage, such as tryptic digest, or by chemical cleavage, e.g. with CNBr, or it can be chemically synthesized using methods well known to the person skilled in the art.

The cells used in step (a) of the method are preferably cells carrying an intact Toll-like receptor 4. However, since it is possible that in man the Toll-like receptor 2 is the hsp60 receptor, also cells carrying such a receptor may be used. Preferably, the cells used in the method are lymphatic cells and more preferably macrophages, e.g. human or mouse macrophages. Examples for cells which can be used in such a method are J774 cells, i.e. the mouse macrophage cell line J774 A.1, which can be purchased from the German Collection of Microorganisms and Cell Cultures (DSMZ, Braunschweig, Germany) or bone marrow-derived macrophages of mouse strains C57BL/6JBom and C3H/HeN, which can be purchased from Breeding & Research Center A/S (Bomholtgård, Ry, Denmark) and from Charles River (Sulzfeld, Germany), respectively. The preparation of such bone-marrow derived macrophages is well known to the person skilled in the art and is also described in Example 1.

As a negative control bone-marrow-derived cells of C3H/HeJ mice, which can be purchased from Charles River (Sulzfeld, Germany), can be used. These macrophages express a functionally defective Toll-like receptor 4 membrane protein and do not respond to hsp60 by the production of TNFα and NO.

The present method is, however, not only useful for identifying fragments of hsp6O having the ability to elicit a pro-inflammatory immune response, but may also be employed for characterizing and/or screening the molecules.

The present invention furthermore relates to a method for the preparation of a pharmaceutical composition comprising steps (a) and (b) of the method described above and the step of formulating the fragment of hsp60 identified in step (a) as having the ability to elicit a pro-inflammatory response into a pharmaceutical composition.

The formulation of the fragment into a pharmaceutical composition may further comprise the addition of a pharmaceutically acceptable carrier, excipient and/or diluent. With the help of the described method, it is now possible to identify smaller fragments of hsp60 which still have the ability to elicit a pro-inflammatory immune response. With the help of such smaller fragments and by identifying those regions of the hsp60 protein which interact with the Toll-like receptor 4 complex it is in turn possible to design variants of these fragments which can act as antagonists of hsp60 thereby reducing or preventing the induction of a pro-inflammatory immune response by hsp60.

Thus, the present invention also relates to variants and derivatives of the hsp60 fragments according to the invention which have the ability to act as an antagonist of hsp60, in particular insofar as they can reduce or prevent the induction of a pro-inflammatory immune response by hsp60. In this context an antagonist is preferably a peptide or peptidomimetic which reduces or prevents such a response.

The present invention also provides a method for identifying, screening and/or characterizing compounds which can act as antagonists of hsp60 insofar as they can reduce or prevent the induction of a pro-inflammatory response by hsp60, wherein this method comprises the steps of

contacting cells which carry on their surface an intact Toll-like receptor complex with hsp60 or a fragment thereof having the ability to induce a pro-inflammatory response in the presence and in the absence of the compound to be tested; and

determining whether the presence of the compound has an effect on the production of a pro-inflammatory mediator or effector,

wherein a reduction or prevention of the production of the pro-inflammatory mediator or effector is indicative of the ability of the compound to act as an antagonist of hsp60.

Currently the methods of the invention are exemplified hereinbelow using the production of TNFα and/or NO of the cells in the assays.

With respect to the cells mentioned in step (a) of the method the same applies as already set forth above in connection with the method for identifying fragments of hsp60 having the ability to elicit a pro-inflammatory immune response. The fragment of hsp60 mentioned in step (a) of the method can be any fragment of hsp60 according to the invention having the ability to elicit a pro-inflammatory immune response.

The determination of the amount of TNFα an/or NO produced by the cells can be carried out as described above and in the Examples. The compound to be tested can be any possible compound, e.g. variants and derivatives of fragments of hsp60 as described above, inorganic or organic compounds, antibodies, e.g. monoclonal or polyclonal antibodies, antibody fragments, such as Fab fragments, soluble Toll-like receptor 4 or Toll-like receptor 2 or fragments or complexes of these receptors, etc.

The present invention furthermore relates to a method for the preparation of a pharmaceutical composition comprising the steps (a) and (b) of the method described above and the step of formulating the compound identified in step (b) as being an antagonist of hsp60 into a pharmaceutical composition.

The formulation of the compound into a pharmaceutical composition might further comprise the addition of a pharmaceutically acceptable carrier, excipient and/or diluent.

The methods for identifying, screening and/or characterizing fragments of hsp60 having the ability to elicit a pro-inflammatory immune response or for identifying compounds which can act as antagonists of hsp60 according to the present invention may also comprise so-called high-throughput screening methods and similar approaches carried out using 96-well, 384-well, 1536-well (and other) commercially available plates (see, inter alia, Spencer, Biotechnol. Bioeng. 61 (1998), 61-67). These methods to be employed in accordance with the present invention comprise, but are not limited to, homogenous optical density readouts (for example for NO and TNFα) in high-throughput screenings (see, e.g. Pope, DDT 4 (1999), 350-362). Cell-based assays, as described hereinabove and in the appended Examples may be combined with detection techniques, like, inter alia, scintillation counting, fluorescence measurement, luminescence measurements or the measurement of absorbance signals. Therefore, the methods described hereinabove and in the appended Examples can easily be adapted to quantitative and/or qualitative screening methods, inducing high-throughput and ultra high-throughput screening methods.

Furthermore, the present invention relates to a pharmaceutical composition comprising a variant, derivative or analog of a fragment according to the invention, which can act as an antagonist of hsp60, and, optionally a pharmaceutically acceptable carrier. The term “antagonist of hsp60” in this respect preferably means that these molecules are able to reduce or prevent the induction of a pro-inflammatory immune response by hsp60.

Finally, the present invention also relates to the use of a variant, derivative or analog of a fragment according to the invention which can act as an antagonist of hsp60 for the preparation of a pharmaceutical composition for the treatment of an inflammatory disease, such as an autoimmune disease (including but not limited to diabetes, multiple sclerosis, rheumatoid arthritis, myasthenia gravis and systemic lupus erythematosis), chronic inflammation (including but not limited to atherosclerosis, inflammatory bowel disease, reactive arthritis), chronic infections, graft rejection, gingivitis, gastritis, ulcer, thrombosis, allergy and acute infections.

Terminology and Definitions:

In this context the term “hsp60 protein” refers to a heat shock protein, i.e. a protein the expression of which is up-regulated in cells when the cells are confronted with, inter alia, a sudden increase in temperature or when they are confronted with other intercellular, intracellular or extracellular stresses. Furthermore, “hsp60 protein” refers to a mitochondrial chaperon. In particular, the term “hsp60 protein” means a protein having a molecular weight of about 60 kD. Preferably, the term “hsp60 protein” means the human hsp60 protein, preferably the protein having the amino acid sequence as disclosed in Jindal et al. (Mol. Cell Biol. 9 (1989), 2279).

For the purpose of the present specification and claims, the term “human hsp60” is intended to comprehend not only the 60 kD human heat shock protein, but also any other related molecule found in the human serum which cross-reacts with polyclonal antibodies raised against a 60 kD heat shock protein of any species. This definition is specifically intended to include, although it is not limited to, the 65 kD, 30 kD, 25 kD and 47 kD proteins which have already been discovered.

The human hsp60 molecule was formerly designated hsp65, but is now designated hsp60 in view of more accurate molecular weight information; by either designation, the protein is the same.

The term “fragment” in the context of the present invention means a part of the hsp60 protein which comprises at least 5, more preferably at least 10, even more preferably al least 20 and most preferably at least 30 consecutive amino acids in a sequence as it occurs in a native hsp60 protein or to a part of an hsp60 protein which may be obtained by cleaving an hsp60 protein, e.g., enzymatically or by other means. In this case the fragment does not necessarily have to contain only a consecutive amino acid sequence, but may contain two or more of such sequences which can be linked to each other, e.g., by disulfide bonds, by peptide bonds or by any other covalent bond.

The term “a molecular weight of about 20 kD or less” in the context of the present invention means a molecular weight of less than 22 kD preferably of less than 21 kD and most preferably of less than 20 kD, in particular when determined by mass spectra or by SDS page under reducing conditions. In a preferred embodiment the fragment has a molecular weight of less than 15 kD, more preferably of less than 10 kD and even more preferably of less than 5 kD.

The term “ability of hsp60 to induce a pro-inflammatory response” in the context of the present invention means in particular the ability to elicit a pro-inflammatory response in the cells of the innate immune system. This can be measured, e.g., by the induction of TNFα and/or NO formation in macrophages, e.g., macrophages of C3H/HeN or C57BL/6 mice or J774 cells (Chen et al., J. Immunol. 162 (1999), 3212), wherein the induction/increase of TNFα and/or NO formation indicates the ability to induce a pro-inflammatory immune response. The determination of the induction of a pro-inflammatory response can, in particular, be carried out as described in Chen et al. (loc. cit.) or in the Examples. Accordingly, the induction of TNFα can be determined by determining the amount of TNFα produced by macrophages before and after exposure to the respective protein, e.g., by using a sandwich ELISA as described in Chen et al. (J. Immunol. 162 (1999), 3212) and quantifying TNFα by using a standard curve obtained, e.g., with the recombinant cytokine (Genzyme, Kent, UK) versus medium alone as blank. The induction of NO formation can be determined, e.g., by measuring the amount of NO released by macrophages by determining the concentration of nitrite (NO₂⁻) accumulated in the culture supernatant using the colorimetric Griess reaction as described in Chen et al. (loc. cit.).

The term “antagonist of hsp60” in the context of the present invention preferably means that these molecules are able to reduce or prevent the induction of a pro-inflammatory immune response by hsp60. This capacity can be determined by using the same assay as described above for determining whether a fragment of hsp60 has the capacity to induce a pro-inflammatory immune response with the exception that, as inductor, e.g., native hsp60 is used and that the variant or derivative to be tested is added and the effect on the production of a pro-inflammatory mediator effector as exemplified by TNFα and/or NO is measured with a decrease or inhibition of the pro-inflammatory mediator or effector (e.g., TNFα and/or NO) production being indicative of the capability of the tested molecule to act as an antagonist of hsp60.

For determining whether a protein, protein fragment and/or peptide induces a pro-inflammatory response, the cells, i.e. macrophages, are for example seeded in a 96 well flat-bottom microtiter plate (2×10⁵ cells in 200 μl per well). After 24 h of preincubation (37° C., 5% CO₂) the protein, protein fragment and/or peptide to be tested is added in various concentrations to the cultures and incubation is continued for different time intervals before measuring the production of TNFα and NO.

As used herein “peptide” indicates a sequence of amino acids linked by peptide bonds. The peptide analogs of this invention comprise a sequence of 5 to 30 amino acid residues, preferably 5 to 24 residues, more preferably 7 to 18 amino acids, each residue being characterized by having an amino and a carboxy terminus.

The term “variant” means that such a molecule has the amino acid sequence of a fragment according to the invention except for one or more amino acid changes. Such variants can be produced, e.g., by mutating the corresponding DNA sequence encoding the corresponding fragment of human hsp60 by point directed mutagenesis, by expressing the thus mutated DNA in an appropriate host cell and by recovering the produced (poly)peptide from the cell culture. Methods for generating such variants and for producing them, e.g. recombinant DNA techniques, are well known to the person skilled in the art. The term “variant”, however, also comprises naturally occurring molecules as well as molecules which may be produced by the recombinant techniques

The term “derivative” means that a fragment according to the invention is modified in such a way that it is no longer capable of inducing a pro-inflammatory immune response but can act as an antagonist of hsp60 in this regard. Such modifications include, e.g., modifications of side chains of one or more amino acid residues of the fragment. In the context of this invention, the term “derivative” comprises therefore “derivative” of fragments of hsp60 which may, preferably, comprise the same three dimensional structure but are chemically and/or physicochemically modified. Furthermore, modification techniques comprise genetic and/or molecular biology techniques. Therefore, the “derivatives” can be produced by mutagenesis techniques or by (side) directed synthesis. Furthermore, techniques well known in the art can be employed to detect and/or characterize such derivatives. These techniques comprise, inter alia, peptidomimetics (al-Obeidi, Mol. Biotechnol. 9 (1998), 205-223; Bohm, J. Com. Aided Mol. Des. 10 (1996), 265-272) or the use of combinatorial libraries (see, e.g., Dorner, Bioorg. Med. Chem. 4 (1996), 705-715). Furthermore, the three-dimensional and/or crystallographic structure of the hsp60 fragment of the present invention can be employed to deduce and/or to generate the “derivatives”. The design of appropriate “derivatives” may be computer assisted.

The term “analog” relates to peptide analogs, which are characterized in that they incorporate novel building units with bridging groups attached to the alpha nitrogens of alpha amino acids. Specifically, these compounds are backbone cyclized analogs comprising a peptide sequence of preferably 5 to 30 amino acids that incorporates at least one building unit, the building unit containing one nitrogen atom of the peptide backbone connected to a bridging group comprising an amide, thioether, thioester or disulfide, wherein the at least one building unit is connected via the bridging group to form a cyclic structure with a moiety selected from the group consisting of a second building unit, the side chain of an amino acid residue of the sequence or the terminal amino acid residue. More preferably, the peptide sequence incorporates 5-24 amino acids, still more preferably it incorporates 7-18 amino acids.

The term “building unit” indicates an N^{α} derivatized a amino acid of the general Formula No. 1:

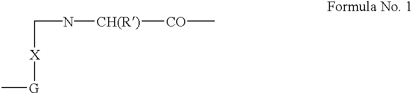

wherein X is a spacer group selected from the group consisting of alkylene, substituted alkylene, arylene, cycloalkylene and substituted cycloalkylene; R′ is an amino acid side chain, optionally bound with a specific protecting group; and G is a functional group selected from the group consisting of amines, thiols, alcohols, carboxylic acids and esters, and alkyl halides; which is incorporated into the peptide sequence and subsequently selectively cyclized via the functional group G with one of the side chains of the amino acids in the peptide sequence or with another ω-functionalized amino acid derivative. The methodology for producing the building units is described in international patent applications published as WO 95/33765 and WO 98/04583 and in U.S. Pat. Nos. 5,770,687 and 5,883,293 all of which are expressly incorporated herein by reference thereto as if set forth herein in their entirety.

The building units are abbreviated by the three letter code of the corresponding modified amino acid followed by the type of reactive group (N for amine, C for carboxyl), and an indication of the number of spacing methylene groups. For example, Gly-C2 describes a modified Gly residue with a carboxyl reactive group and a two carbon methylene spacer, and Phe-N3 designates a modified phenylalanine group with an amino reactive group and a three carbon methylene spacer.

Certain abbreviations are used herein to describe this invention and the manner of making and using it.

For instance, 2Abu refers to 2-aminobutyric acid, Alloc refer to allyloxycarbonyl, Boc refers to the t-butyloxycarbonyl radical, DIEA refers to diisopropyl-ethyl amine, EDT refers to ethanedithiol, Fmoc refers to the fluorenylmethoxycarbonyl radical, HBTU refers to 1-hydroxybenztriazolyltetramethyl-uronium hexafluorophosphate, HOBT refers to 1-hydroxybenzotriazole, HPLC refers to high pressure liquid chromatography, hsp refers to heat shock protein, IDDM refers to Insulin-dependent Diabetes Mellitus, kD referees to Kilo Dalton, MPS refers to Multiple parallel synthesis, MS refers to mass spectrometry, NMM refers to N-methylmorpholine, NMP refers to 1-methyl-2-pyrolidonone, NO refers to nitric oxide, PyBOP refers to Benzotriazole-1-yl-oxy-tris-pyrrolidino-phosphonium hexafluorophosphate, PyBrOP refers to Bromo-tris-pyrrolidino-phosphonium hexafluorophosphate, TFA refers to trifluoroacetic acid, and TNF refers to tumor necrosis factor.

The amino acids used in this invention are those which are available commercially or are available by routine synthetic methods. Certain residues may require special methods for incorporation into the peptide, and either sequential, divergent or convergent synthetic approaches to the peptide sequence are useful in this invention. Natural coded amino acids and their derivatives are represented by three-letter codes according to IUPAC conventions. When there is no indication, the L isomer was used. The D isomers are indicated by “D” before the residue abbreviation.

Conservative substitution of amino acids as known to those skilled in the art are within the scope of the present invention. Conservative amino acid substitutions includes replacement of one amino acid with another having the same type of functional group or side chain e.g. aliphatic, aromatic, positively charged, negatively charged. These substitutions also include replacement of Phe residues with N-Methyl-Phe residues for increasing the bio-availability of the compound and conjugation of mono- and disaccharides moieties at the amino terminus for increasing oral bio-availability (Nelson-Piercy et al. J. Clin. Endocrinol. And Metab. 78:329, 1994), or other such substitutions as may enhance oral bioavailability, penetration into the central nervous system, targeting to specific cell populations and the like.

Description of Preferred Analogs

The currently preferred fragments, variants, derivatives and analogs of hsp60 of the present invention are now disclosed.

Preferred embodiments are derived from the sequence of the human hsp60. Additional preferred embodiments are derived from the sequence of the bacterial hsp60.

Preferred embodiments according to the present invention includes variants, derivatives and analogs of fragments having a sequence selected from the group consisting of:

a. Residues 126-160 of human hsp60. A more preferred portion of this region spans residues 126-156 of human hsp60;

b. Residues 140-192 of human hsp60. More preferred portion of this region spans residues 157-191, 158-192, 158-191, 161-191, and 161-192 of human hsp60;

c. Residues 220-250 of human hsp60. A most preferred portion of this region spans residues 222-249 of human hsp60;

d. Residues 269-290 of human hsp60;

e. Residues 310-344 of human hsp60. Preferred portion of this region spans residues 315-344 of human hsp60;

f. Residues 390-421 of human hsp60;

g. A currently most preferred embodiment according to the present invention comprises derivatives, variants and peptide analogs of residues 350-400 of the human hsp60.

Additional most preferred peptide analogs according to the present invention are;

i. Tyr-Phe-Ile-Asn-Thr-Ser-Lys-Gly-Gln-Lys corresponding to residues 227-236 of human hsp60;

ii. Lys-Ala-Gln-Ile-Glu-Lys-Arg-Ile-Gln-Glu corresponding to residues 364-373 of human hsp60;

iii. Lys-Arg-Ile-Gln-Glu-Ile-Ile-Glu-Gln-Leu corresponding to residues 369-378 of human hsp60;

iv. Ile-Gln-Glu-Ile-Ile-Glu-Gln-Leu-Asp-Val corresponding to residues 371-380 of human hsp60;

v. Gln-Glu-Ile-Ile-Glu-Gln-Leu-Asp-Val-Thr corresponding to residues 372-381 of human hsp60;

vi. Val-Leu-Lys-Val-Gly-Gly-Thr-Ser-Asp-Val corresponding to residues 403-412 of human hsp60;

vii. Lys-Val-Gly-Gly-Thr-Ser-Asp-Val-Glu-Val corresponding to residues 405-414 of human hsp60.

Additional more preferred derivatives, variants and peptides analogs according to the present invention are based on residues 364-381 of the human hsp60 sequence: Lys-Ala-Gln-Ile-Glu-Lys-Arg-Ile-Gln-Glu-Ile-Ile-Glu-Gln-Leu-Asp-Val-Thr. The preferred peptide analogs of this sequence are peptides of 10-18 residues with optionally at least one residue replaced with an Alanine. The most preferred peptide analogs are:

i. Lys-Ala-Gln-Ile-Glu-Lys-Arg-Ile-Gln-Glu-Ile-Ile-Ala-Gln-Leu corresponding to residues 367-381 of human hsp60;

ii. Lys-Arg-Ala-Gln-Glu-Ile-Ile-Glu-Ala-Leu corresponding to residues 372-381 of human hsp60.

Another preferred embodiment according to the present invention comprises derivatives, variant and peptide analogs of residues 403-412 of the human hsp60. The most preferred analogs of this region include the following sequences:

Val-Leu-Lys-Val-Gly-Gly-Thr-Ser-Asp-Val-Glu-Val-Asn;

Ala-Val-Leu-Lys-Val-Gly-Gly-Thr-Ser-Asp-Val-Glu-Val;

Val-Ala-Val-Leu-Lys-Val-Gly-Gly-Thr-Ser-Asp-Val-Val;

Gly-Val-Ala-Val-Leu-Lys-Val-Gly-Gly-Thr-Ser-Asp-Val.

Yet other more preferred analogs according to the present invention are backbone cyclized peptide derivatives, variants and analogs of residues 408-416 of the human hsp60. These analogs optionally comprise Gln, Ser, Thr, Ala, Leu or 2Abu residue at position 415 instead of Asn. Most preferred analogs of this sequence are nonapeptides comprising two bridging groups at positions: 408-414, 409-416, 408-416, and 409-414 which are connected to form cyclic structure. Other preferred backbone cyclized analogs have 5-18 residues with at least one building unit connected to a terminal, a side chain or another building unit to form a cyclic peptide analog.

All the sequences listed above and in the examples as useful per se and may also be used as a basis for the design and synthesis of derivatives, variants and analogs with improved properties.

General Methods

General Method for Synthesis, Purification and Characterization of Peptides:

Synthesis:

Resin: 1 g Rink amide or Tenta-gel resin, with loading of 0.2-0.7 mmol/gr.

Fmoc-deprotection: With 7 mL of 20% piperidine in NMP. Twice for 15 minutes following 5 washes with 10 ml NMP for 2 minutes with shaking.

Couplings:

1. Regular couplings (coupling to simple amino acids): with a solution containing 3 equivalents amino acid, 3 equivalents PyBroP and 6 equivalents of DIEA in 7 ml NMP. For 0.5-2 hours with shaking. Coupling is monitored by ninhydrine test and repeated until the ninhydrine solution become yellow.

2. Coupling of His and Asn with a solution containing 5 equivalents DIC and 5 equivalents HOBT in 10 ml dimethyl formamide.

3. Coupling to Gly building units: with a solution containing 3 equivalents amino acid, 3 equivalents PyBroP and 6 equivalents DIEA in 7 ml NMP. Twice for 1-4 hours with shaking.

4. Coupling to building units which are not Gly: with a solution containing 5 equivalents amino acid, 1.5 equivalents triphosgen and 13 equivalents collidine in 15 ml dioxane or THF. Three times for 40 minutes at 60° C. with shaking.

In those embodiments where backbone cyclized peptide analogs are synthesized, orthogonal protection is required. According to a currently preferred embodiment Allyl/Alloc deprotection and cyclization is performed as follows:

Removal of the Allyl and Alloc protecting groups of the building units: with 1.5 equivalents per peptide of Pd(PPh3)₄ in 30 ml dichloromethane containing 5% acetic acid and 2.5% NMM. For 1-4 hours with shaking.

Cyclization: with a solution containing 3 equivalents PyBOP and 6 equivalents DIEA in 7 ml NMP. For 0.5-2 hours with shaking. Cyclization is monitored by ninhydrine test and repeated if necessary.

Cleavage: with 82%-95% TFA supplemented with scavengers: 1-15% H₂O, 1-5% TIS and 1-5% EDT.

Purification:

An individual purification method for each backbone cyclic peptide is developed on analytical HPLC to give the maximum isolation of the cyclic peptide from other crude components. The analytical method is usually performed using a C-18 Vydac column 250×4.6 mm as the stationary phase and water/ACN containing 0.1% TFA mixture gradient.

The preparative method is designed by implying the analytical separation method on the 2″ C.-18 Vydac preparative method. During the purification process, the peak containing the cyclic peptide is collected using a semi-automated fraction collector. The collected fractions are injected to the analytical HPLC for purity check. The pure fractions are combined and lyophilized.

Characterization:

The combined pure lyophilized material is analyzed for purity by HPLC, MS and capillary electrophoresis and by amino acid analysis for peptide content and amino acid ratio determination.

General Method for Synthesis, Purification and Characterization of Libraries in Multiple Parallel Synthesis (MPS) Format:

The MPS procedure is used as the routine peptide development procedure. Individual peptides, or groups of a few peptides, are synthesized in 96-wells microtiter plates equipped with filters that allow passage of solvent but not of solid phase matrix. A simple and efficient valve apparatus that enable simultaneous closing and opening of all the valves (produced by Millipore) is used. The system utilizes an approach in which each well is equipped with a solvent permeable membrane at the bottom that does not pass particles above a certain size. The process allows to place resin in the wells, perform reaction in solvent, and remove the solvent from all the wells simultaneously by applying vacuum. These special plates, which are available in the standard 96 well format allow the parallel synthesis of 96 peptides simultaneously. The synthesis scale of the procedure is in the range of 1-5 μmole per well. Following purification by C 18 reverse phase columns (SepPak purification), which is also carried in the standard 96 well format, the peptides are routinely dissolved in 1 ml of water to yield a theoretical crude concentration of 1-5 mM (depending of synthesis scale). Monitoring of chemical quality of the resulting peptides is performed by ESI-MS analysis. Analysis of several plates prepared on different occasions by different operators indicated a general success rate of about 80% as judged by the presence of the desired peptide mass in the crude preparation. Further analysis of a peptides from MPS is carried out by LC-MS. The analysis revealed crude peptide quality similar to crud preparations of peptides synthesized individually in large scale. Different steps or the complete process is now performed automatically using automatic peptide synthesizers. According to the present exemplifications, the peptides are currently synthesized automatically using the ACT 396 of Advanced ChemTech, and the heating device Lab Tech 4 of Advanced ChemTech.

Detailed Procedure for Synthesis in MPS Format:

For capacity of 6 μmole 10 mg resin with a substitution of 0.6 mmol/gr is used.

Fmoc deprotection: To each well 500 μl of 5% piperidine in NMP are added twice. The reaction shacked for 15 min. The NMP is removed by suction.

Washing after Fmoc deprotection: the resin is washed by placing 600 μl NMP into each well followed by evacuation of the solution by steam of nitrogen. The washing process is repeated 4 times.

Coupling Using HBTU:

Well capacity: 6 μmol

Amount of amino acid per coupling per well: 30 μmol

Amino acid in NMP concentration: 0.2 M

Amino acid volume used: 150 μl

HBTU amount: 30 μmol

HBTU concentration: 0.2 M

HBTU volume used: 150 μl

DIEA added: 150 μl of 0.4 M in NMP

Total reaction volume: 450 μl

The amino acids are dissolved in a solution of HOBT in NMP. The resin is washed by placing 600 μl NMP into each well followed by evacuation of the solution by steam of nitrogen. The washing process is repeated 4 times. The coupling reaction is repeated twice for 1 hour.

Coupling Using Mukayama Reagent (Performed Only in Certain Situations):

Amino acid solution at 650 mM-40 μl

Mukayama reagent at 111 mg/ml-60 μl

Collidine added per well-15 μl

The same procedure as for coupling with PyBroP. Reaction temperature 50° C., reaction time: first coupling 4 h, second coupling 16 h.

In Embodiments Wherein Backbone Cyclized Peptides are Synthesized, Allyl/Alloc Deprotection and Cyclization is Performed as Following:

Allyl Alloc deprotection: this step is performed after completing the assembly, by addition of 180 μl solution of 1.5 g Pd(PPh₃)₄ in 20 ml CH₂Cl₂ containing 5% acetic acid +2.5% NMM.

Cyclization: this step is performed by addition of 100 μl solution of PyBoP in NMP+DIEA.

Cleavage of the peptide from the resin and SepPak purification: After final Fmoc deprotection the resin is transferred into a deep well microtiter plate, to each well 300 μl of TFA solution containing 2.5% TIS, 2.5% H₂O, 2.5% EDT are added. Removal of the TFA is performed by lyophilization. After cleavage the peptides are purified by SepPak.

Biological Screening of Fragments, Derivatives, Variants and Analogs of Hsp60

1. Identifying Fragments of Hsp60 Having the Ability to Elicit a Pro-Inflammatory Immune response:

This method comprises the following steps:

contacting cells carrying on their surface an intact Toll-like receptor complex with a fragment of hsp60; and

determining the amount of TNFα and/or NO produced by the cells;

An increase in the production of TNFα and/or NO is indicative for the ability of the fragment of hsp60 to elicit a pro-inflammatory response.

The fragment of hsp60 which is used in step (a) can be any possible fragment of hsp60. Preferably, such a fragment has a length of at least 5 amino acid residues, more preferably of at least 10, even more preferably of at least 20 and most preferably of at least 50 amino acid residues. Such a fragment may be produced, e.g., by cleavage of the hsp60 protein, for example by proteolytic cleavage, such as tryptic digest, or by chemical cleavage, e.g. with CNBr, or it can be chemically synthesized using methods well known to the person skilled in the art.

The cells used in step (a) of the method are preferably cells carrying an intact Toll-like receptor 4. However, since it is possible that in man the Toll-like receptor 2 is the hsp60 receptor, also cells carrying such a receptor may be used. Preferably, the cells used in the method are lymphatic cells and more preferably macrophages, e.g. human or mouse macrophages. Examples for cells which can be used in such a method are J774 cells, i.e. the mouse macrophage cell line J774 A. 1, which can be purchased from the German Collection of Microorganisms and Cell Cultures (DSMZ, Braunschweig, Germany) or bone marrow-derived macrophages of mouse strains C57BL/6JBom and C3H/HeN, which can be purchased from Breeding & Research Center A/S (Bomholtgård, Ry, Denmark) and from Charles River (Sulzfeld, Germany), respectively. The preparation of such bone-marrow derived macrophages is well known to the person skilled in the art and is also described in Example 1.

As a negative control bone-marrow-derived cells of C3H/HeJ mice, which can be purchased from Charles River (Sulzfeld, Germany), can be used. These macrophages express a functionally defective Toll-like receptor 4 membrane protein and do not respond to hsp60 by the production of TNFα and NO.

For determining whether a protein, protein fragment and/or peptide induces a pro-inflammatory response, the cells, i.e. macrophages, are for example seeded in a 96 well flat-bottom microtiter plate (2×10⁵ cells in 200 μl per well). After 24 h of preincubation (37° C., 5% CO₂) the protein, protein fragment and/or peptide to be tested is added in various concentrations to the cultures and incubation is continued for different time intervals before measuring the production of TNFα and NO.

2. Identifying, Screening and/or Characterizing Compounds Which can Act as Antagonists of hsp60:

The capacity for reduction or preventing the induction of a pro-inflammatory immune response by hsp60 can be determined by using method no. 1 as described above with the exception that, as inductor, e.g., native hsp60 is used and that the variant or derivative to be tested is added and the effect on the production of TNFα and/or NO is measured with a decrease or inhibition of TNFα and/or NO production being indicative of the capability of the tested molecule to act as an antagonist of hsp60.

The method comprises the following steps:

contacting cells which carry on their surface an intact Toll-like receptor complex with hsp60 or a fragment thereof having the ability to induce a pro-inflammatory response in the presence and in the absence of the compound to be tested; and

determining whether the presence of the compound has an effect on the production of TNFα and/or NO of the cells,

wherein a reduction or prevention of the production of TNFα and/or NO is indicative of the ability of the compound to act as an antagonist of hsp60.

With respect to the cells mentioned in step (a) of the method the same applies as in method 1 above. The fragment of hsp60 mentioned in step (a) of the method can be any fragment of hsp60 according to the invention having the ability to elicit a pro-inflammatory immune response.

The determination of the amount of TNFα an/or NO produced by the cells can be carried out as described above and in the Examples.

The compound to be tested can be any possible compound, e.g. variants and derivatives of fragments of hsp60 as described above, inorganic or organic compounds, antibodies, e.g. monoclonal or polyclonal antibodies, antibody fragments, such as Fab fragments, soluble Toll-like receptor 4 or Toll-like receptor 2 or fragments or complexes of these receptors, etc.

3. Methods for Measuring the Induction of TNFα and/or NO Formation:

The induction of TNFα and/or NO formation can be measured in macrophages, e.g., macrophages of C3H/HeN or C57BL/6 mice or J774 cells (Chen et al., J. Immunol. 162 (1999), 3212), wherein the induction/increase of TNFα and/or NO formation indicates the ability to induce a pro-inflammatory immune response. The determination of the induction of a pro-inflammatory response can, in particular, be carried out as described in Chen et al. (loc. cit.) or in the Examples. Accordingly, the induction of TNFα can be determined by determining the amount of TNFα produced by macrophages before and after exposure to the respective protein, e.g., by using a sandwich ELISA as described in Chen et al. (J. Immunol. 162 (1999), 3212) and quantifying TNFα by using a standard curve obtained, e.g., with the recombinant cytokine (Genzyme, Kent, UK) versus medium alone as blank. The induction of NO formation can be determined, e.g., by measuring the amount of NO released by macrophages by determining the concentration of nitrite (NO₂⁻) accumulated in the culture supernatant using the colorimetric Griess reaction as described in Chen et al. (loc. cit.).

In addition, fragments, variants, derivatives and analogs of hsp60 may be screened for T-cell proliferation responses, influence on insulitis and diabetes in mice models, and peptide stability as described in PCT applications WO 96/19236 and WO 97/01959.

The methods for identifying, screening and/or characterizing fragments of hsp60 having the ability to elicit a pro-inflammatory immune response or for identifying compounds which can act as antagonists of hsp60 according to the present invention may also comprise so-called high-throughput screening methods and similar approaches carried out using 96-well, 384-well, 1536-well (and other) commercially available plates (see, inter alia, Spencer, Biotechnol. Bioeng. 61 (1998), 61-67). These methods to be employed in accordance with the present invention comprise, but are not limited to, homogenous optical density readouts (for example for NO and TNFα) in high-throughput screenings (see, e.g. Pope, DDT 4 (1999), 350-362). Cell-based assays, as described hereinabove and in the appended Examples may be combined with detection techniques, like, inter alia, scintillation counting, fluorescence measurement, luminescence measurements or the measurement of absorbance signals. Therefore, the methods described hereinabove and in the appended Examples can easily be adapted to quantitative and/or qualitative screening methods, inducing high-throughput and ultra high-throughput screening methods.

Fragments, variants and derivatives of heat shock proteins are constructed based in part on the sequences of a number of known heat shock proteins, or based on previously unknown novel sequences, are presented in the examples below. The following examples are intended to illustrate how to make and use the compounds and methods of this invention and are in no way to be construed as a limitation.

### EXAMPLES

The following Examples further illustrate the invention.

### Example 1

### Identification of the Toll-Like Receptor 4 Complex as a Possible Ligand for Hsp60

Recombinant human hsp60 was obtained from StressGen Biotechnologies (Victoria, Canada). The two immunostimulatory oligonucleotides 5′-ACC GAT AAC GTT GCC GGT GAC G-3′(Pal+) (Yamamoto et al., Microbiol. Immunol. 38 (1994), 831) and 5′-TCC ATG ACG TTC CTG ATG CT-3′(ODN1668) (Hücker et al., EMBO J. 17 (1998), 6230; Sparwasser et al., J. Immunol. 27 (1997), 1671) containing a CpG motif and the corresponding non-stimulatory oligonucleotide 5′-TCC ATG AGC TTC CTG ATG CT -3′ (ODN1720) (Häcker et al., EMBO J. 17 (1998), 6230; Sparwasser et al., J. Immunol. 27 (1997), 1671) lacking a CpG motif were purchased from Life Technologies (Karlsruhe, Germany). Lipopolysaccharide (LPS) from Escherichia coli B 0.26 was obtained from Sigma (Deisenhofen, Germany).

For the preparation of Mouse bone marrow-derived macrophages C3H/HeN and C3H/HeJ mice were purchased from Charles River (Sulzfeld, Germany) and C57BL/6JBom mice were purchased from Breeding & Research Center A/S (Bomholtgård, Ry, Denmark). Bone marrow cells were obtained by flushing femurs and tibias of 8-12 weeks old mice with ice-cold PBS. After washing 2.5×10⁶ bone marrow cells were incubated (37° C., 5% CO₂) in tissue culture dishes with 10 ml of Pluznik medium containing 5% heat-inactivated horse serum, 15% fetal calf serum (FCS, Life Technologies), 15% L929 cell-conditioned medium (Burgess et al., J. Biol. Chem. 260 (1985), 16004) and 65% RPMI 1640 supplemented with ampicillin (25 mg/l), penicillin (120 mg/l), streptomycin (270 mg/l), 1 mM sodium pyruvate, 2 mM L-glutamine, non-essential amino acids (10 ml/l, 100×), 24 mM NaHCO₃, and 10 mM HEPES. After 7 days of cultivation, adherent bone marrow-derived macrophages were detached by incubation with ice-cold Ca⁺⁺-, Mg⁺⁺-free Hank's balanced salt solution (HBSS, Life Technologies) for 10 min followed by two washes with HBSS (500 g, 5 min). By non specific esterase stain (Löffler, Klin. Wschr. 39 (1961), 1220)>98% of cells exhibited macrophage characteristics.

For the stimulation of macrophages cells were seeded in 96 well flat-bottom microtiter plates (Falcon/Becton Dickinson, Franklin Lakes, N.J.) (2×10⁵ cells in 200 μl per well). After 24 h of preincubation (37° C., 5% CO₂) various concentrations of hsp60, LPS and oligonucleotides were added to the cultures and the incubation was continued for different time intervals.

The amounts of TNFα released from the macrophages were quantified by sandwich ELISA (Chen et al., J. Immunol. 162 (1999), 3212). The TNFα was quantified using a standard curve obtained with the recombinant cytokine (Genzyme, Kent, UK) versus medium alone as blank. The results were expressed as pg TNFα per ml.

For the measurement of nitrite production the amount of NO released by macrophages was assessed by determining the concentration of nitrite (NO₂—) accumulated in the culture supernatant using the colorimetric Griess reaction as described previously (Chen et al., loc. cit.). The results show micromoles of NO₂⁻ per ml.

The data obtained are expressed as mean +SD. Statistical analysis was performed using the Student's test, two-sided. Differences were considered statistically significant with p<0.05.

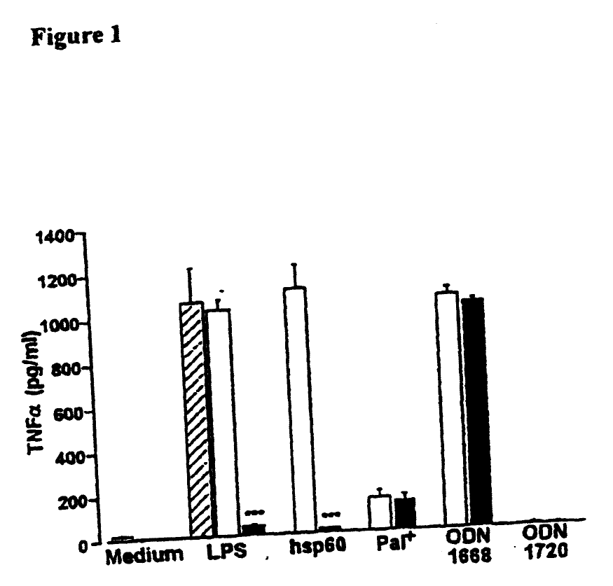

As is evident from FIG. 1, bone marrow-derived macrophages of mouse strains C57BL/6 and C3H/HeN responded to LPS or human hsp60 with rapid secretion of large amounts of TNFα. A parallel study of macrophages from C3H/HeJ mice, which carry a mutant Toll-like receptor 4, showed no response to either LPS or hsp60. However, these cells were not completely refractory to inflammatory stimuli. An oligodeoxynucleotide derived from mycobacterial sequences (ODN1668) and containing a potent immunostimulatory CpG motif induced a strong TNFα response in the LPS non-responder strain. A second CpG containing oligonucleotide (Pal⁺) was less stimulatory while a CpG deficient oligonucleotide (ODN1720) did not provoke a response (FIG. 1).

FIG. 1 shows bone marrow-derived macrophages of C57BL/6 (hatched bars), C3H/HeN (open bars) and C3H/HeJ mice (solid bars) were incubated with medium, LPS (10 ng/ml), hsp60 (10 μg/ml), Pal⁺ (30 μg/ml), ODN1668 (30 μg/ml) or ODN1720 (30 μg/ml). After 6 h the TNFα concentration in the culture supernatant was determined by ELISA. The data represent means +SD of three to four experiments performed in quadruplicates. Significant differences to C3H/HeN (and C57BL/6) macrophages are indicated as ***, p<0.001.

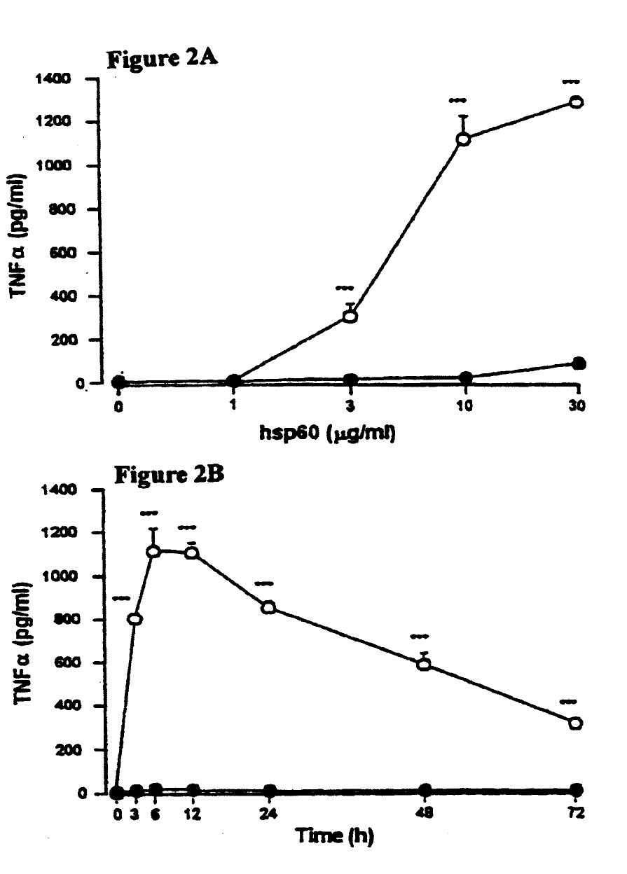

The dose dependence of the response to hsp60 was further analyzed with macrophages from the two C3H strains. A significant TNFα response was obtained in C3H/HeN macrophages with 3 μg/ml hsp60 (0.05 μmol/l) while no such response was seen in C3H/HeJ macrophages even at ten times higher hsp60 levels (FIG. 2A). An analysis of the kinetics revealed peak levels of secreted TNFα between 6-12 h in C3H/HeN macrophages. In the TIr4 defective macrophages TNFα production was absent throughout the observation period of 72 h (FIG. 2B).

FIG. 2 shows bone marrow-derived macrophages of C3H/HeN (open circles) and C3H/HeJ mice (solid circles) were incubated for 6 h with increasing concentrations of hsp60 (A) or they were incubated for different time intervals at a dose of 10 μg/ml hsp60 (B). The TNFα concentrations in the culture supernatant were determined by ELISA. The data represent means +SD from three experiments performed in triplicate. Significant differences to C3H/HeJ derived macrophages are indicated as ***, p<0.001.

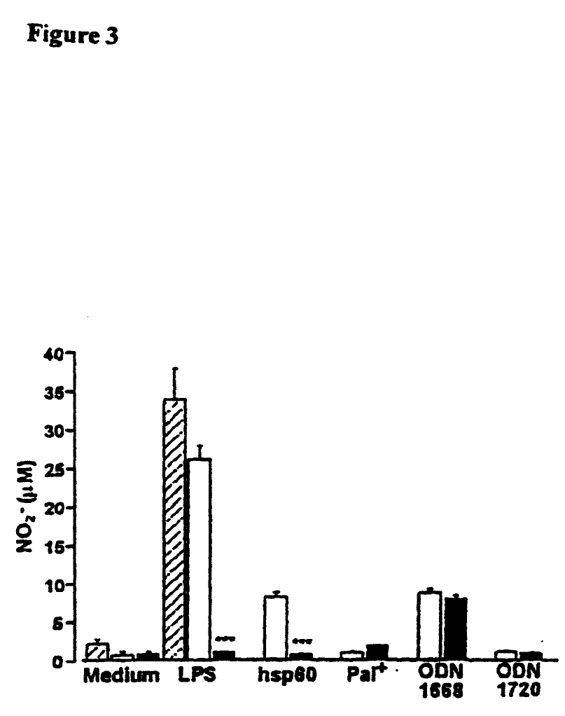

As is evident from FIG. 3 macrophages of C57BL/6, C3H/HeN but not of C3H/HeJ mice responded to LPS challenge with NO production, which indicates that endotoxin induced NO formation is Tlr4 dependent.

FIG. 3 shows bone marrow-derived macrophages of C57BL/6 (hatched bars), C3H/HeN (open bars) and C3H/HeJ mice (solid bars) were incubated with medium, LPS (10 ng/ml), hsp60 (10 μg/ml), Pal+(30 μg/ml), ODN1668 (30 μg/ml) or ODN1720 (30 μg/ml). After 24 h the concentration of nitrite accumulated in the culture supernatant was determined by the Griess reaction. The data represent means +SD of three to four experiments performed in quadruplicates. Significant differences to C3H/HeN (and C57BL/6) macrophages are indicated as ***, p<0.001.

The same outcome was obtained when hsp60 was taken as stimulus, with a complete lack of a nitric oxide response in Tlr4 defective macrophages. In contrast, the two macrophage types showed a very similar NO response when the strongly stimulatory CpG DNA ODN1668 was used as stimulus while the less stimulatory CpG oligonucleotide Pal+failed to induce NO formation, as was the case for the CpG deficient ODN1720 (FIG. 3).

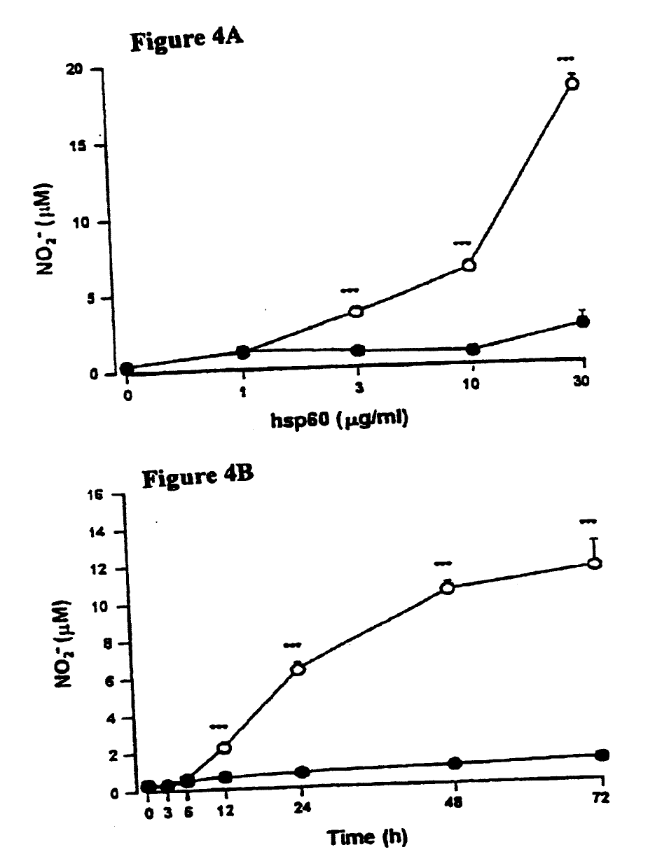

Similar concentrations of hsp60 (3 μg/ml) were required for eliciting NO compared to TNFα production. Increasing the hsp60 concentration tenfold yielded about five times higher nitrite levels in C3H/HeN macrophages while Tlr4 defective macrophages remained completely refractory (FIG. 4A). Nitrite accumulation in the supernatant reached maximum levels between 48 and 72 h of culture. There was no indication of NO production in C3H/HeJ macrophages throughout this period (FIG. 4B).

FIG. 4 shows Bone marrow-derived macrophages of C3H/HeN (open circles) and C3H/HeJ mice (solid circles) were incubated for 24 h with increasing concentrations of hsp60 (A) or they were incubated for different time intervals at a dose of 10 μg/ml hsp60 (B). The concentrations of nitrite accumulated in the culture supernatants were determined by the Griess reaction. The data represent means +SD of three experiments performed in triplicates. Significant differences to C3H/HeJ derived macrophages are indicated as ***, p<0.001.

The above described results suggest that extracellular hsp60 is an endogenous ligand of Tlr4. Compared were bone marrow derived macrophages of two closely related strains, C3H/HeN and C3H/HeJ, differing in the ability to respond to endotoxin with an inflammatory response (Sultzer, Nature 219 (1968), 1253; Watson and Riblet, J. Exp. Med. 140 (1974), 1147). This functional difference was recently identified as being due to a functionally defective Tlr4 membrane protein in C3H/HeJ mice. In the latter strain the C-terminal part of Tlr4 contains a mutation at codon 712 which interferes with LPS induced signaling (Ips^{d}) (Poltorak et al., Science 282 (1998), 2085; Qureshi et al., J. Exp. Med. 189 (1999), 165; Hoshino et al., J. Immunol. 162 (1999), 3749). Interestingly, Tlr4 defective macrophages are not completely non responsive to LPS because endotoxin is still able to stimulate the expression of metalloproteinase-9 (Jin et al., J. Immunol. 162 (1999), 3596).

Indeed, macrophages from C3H/HeJ (Ips^{d}) mice were found clearly refractory to LPS induced TNFα production. Interestingly, inducible NO formation was also found strictly dependent on a functional Tlr4 although signaling requirements differ from that of TNFα (Lowenstein et al., Proc. Natl. Acad. Sci. USA 90 (1993), 9730; Kajimo et al., Science 263 (1994), 1612). Exposure of lps^{d} macrophages to immunostimulatory CpG DNA did stimulate both, TNFα and NO production, which shows that Tlr4 dependent and independent pathways for stimulating innate immune responses co-exist in macrophages. Also these data demonstrate that CpG DNA signaling does not occur via Tlr4.

Taken together, stimulation of TNFα or NO response by human hsp60 was found here as fully dependent on the presence of a functional Tlr4 membrane protein. Thus, the same transmembrane signaling receptor appears to mediate the innate immune response to hsp60 and LPS. Extensive controls were performed in two previous reports of the immunostimulatory activity of extracellular mammalian hsp60 in order to exclude a role of endotoxin contamination (Kolb et al., J. Clin. Invest. 103 (1999), 571; Chen et al., J. Immunol. 162 (1999), 3212). These controls included the use of polymyxin B for neutralization of LPS, or denaturing of protein by heat treatment which suppressed hsp60 but not LPS activity. Also, we were able to repeat the essential findings with an endotoxin-free preparation from another source (from Peptor Ltd., less than 0.1 μEU of endotoxin contamination per μg hsp60, unpublished data). Finally, endotoxin-free bacterial lipoproteins have been recently reported to mediate an innate immune response via the Toll-like receptor-2 pathway (Aliprantis et al., Science 285 (1999), 736; Brighill et al., Science 285 (1998), 736; Hirschfeld et al., J. Immunol. 163 (1999), 2382). These observations underscore that Tlr4 and Tlr2 do not only function as receptors of LPS (Poltorak et al., loc. cit.; Kirschning et al., J. Exp. Med. 188 (1998), 2091; Hirschfeld et al., loc. cit.) but are also involved in the recognition of protein ligands.

The previously described ligands for Toll-like receptors in mammalian cells are of microbial origin, which is in line with a function of these receptors in innate immune responses. It was now found that the chaperone hsp60 is a putative endogenous ligand of Toll-like receptors in mammals. This finding suggests that Toll-like receptors may not only have a function in innate immune defense against microbial pathogens but serve also physiological functions by interacting with endogenous ligands. This is reminiscent of the situation in Drosophila where Toll controls dorsal-ventral patterning with spaetzle serving as endogenous ligand (Marisato and Anderson, Cell 76 (1994), 677), while in adult insects Toll controls the antifungal and antibacterial response (Lemaitre et al., Cell 86 (1996), 973).

It is noteworthy that both, Toll-like receptors and hsp60 are found early in phylogeny and both are of remarkably conserved structure. This indicates that their interaction is relevant and may also occur in more primitive organisms. Mammalian hsp60 usually is sequestered to the cell interior, in accordance with its ability to function as chaperone. However, hsp60 becomes accessible when it is set free during necrosis of tissue cells during inflammation or when hsp60 is partially translocated to the plasma membrane in response to diverse types of stress. It was therefore proposed that autologous hsp60 may serve as danger antigen to the innate immune system (Chen et al., loc. cit.).

The exact mechanism of interaction between mammalian hsp60 and the Tlr4 complex remains to be elucidated. With CD14 and MD-2 two members of the Tlr4 complex have been identified, both of which strongly potentiate LPS responsiveness of Tlr4. LPS appears to bind to Tlr4 via CD 14 as well as independent of CD 14. For human TIr2 direct binding to LPS was demonstrated in vitro and efficient signaling appears to require serum CD14. Similarly, the mechanism of interaction between bacterial lipoproteins and Tlr2 has not yet been determined.

In summary, the pro-inflammatory signaling of human hsp60 was found dependent on a functional Tlr4. This finding suggests the existence of endogenous ligands of the Tlr4 complex, and a role of Toll-like receptors in innate immune discrimination of normal versus stressed or damaged tissue cells.

### Example 2

### Identification of Fragments of Human Hsp60 Capable of Inducing a Pro-Inflammatory Response in Macrophages

1. Enzymatic Proteolysis of Hsp60 by Trypsin:

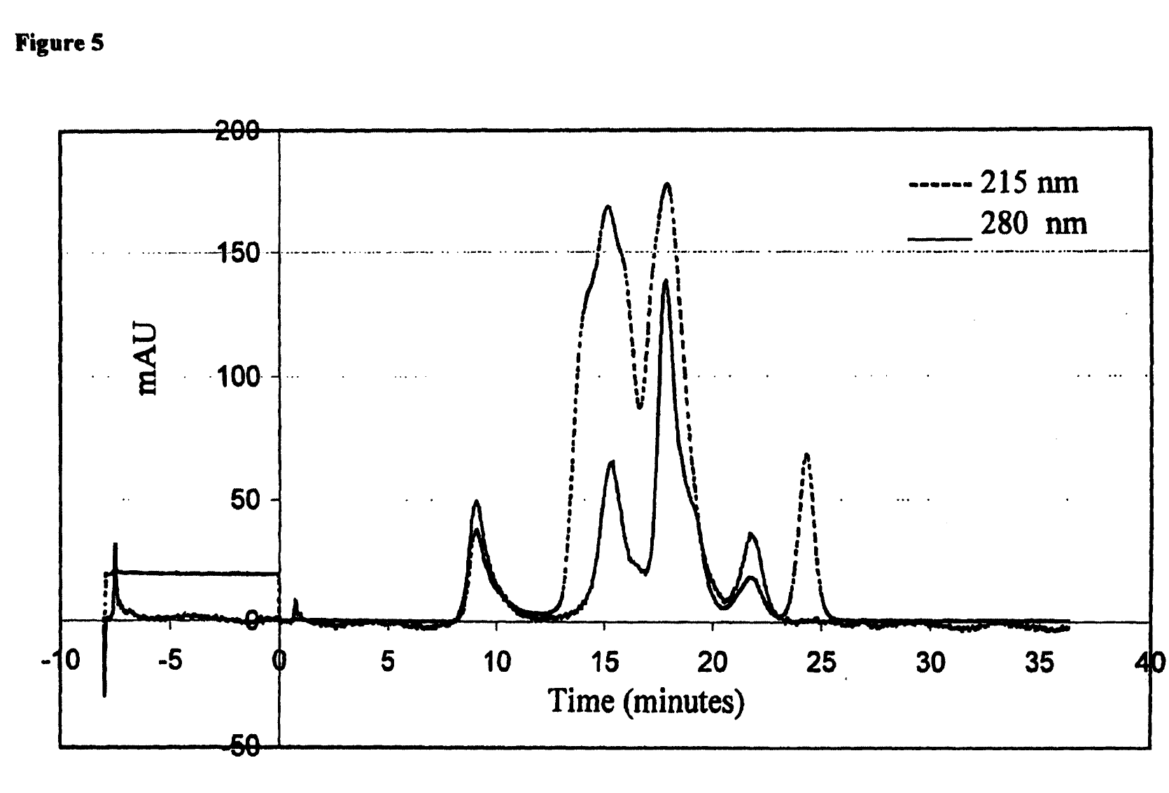

His-tag-hsp60 (hsp60 expressed with six Histidine residues at the N-terminus of the protein), was affinity-purified on a nickel column (the hexa-Histidine is a chelator of nickel). Sixty μl of 4.5 mg/ml in PBS were used to dissolve 50 μg of sequencing grade modified trypsin (Boehringer cat. No. 1418025) in an enzyme: protein weight ratio of 1:54. The mixture was incubated at 37° C. and samples were taken after 0.5, 1.5, 5.4 and 16.5 hours. The samples were stored overnight at 4° C. and then transferred to −20° C. Samples of 5 μl each were boiled with 5 μl loading buffer and run on SDS-PAGE (4-20% gradient for 30 minutes at 200V, BioRad). The 4 fractions were combined together and centrifuged for 1 minute. The supernatant was filtered and then submitted to gel filtration chromatography on Superdex-75 FPLC column with PBS in a rate of 0.5 ml/min. Five clear fractions plus two peak shoulders were obtained as described in FIG. 5. It was found (after similar gel filtration chromatography of hsp60) that the first peak contains intact hsp60. Protein concentration was determined by Micro BSA kit (Pierce). The results are summarized in Table No. 1.

TABLE 1

| | | | | | | | |
|---|---|---|---|---|---|---|---|
| 7 | 6 | 5 | 4 | 3 | 2 | 1 | Fraction |
| 2.5 | 2.5 | 3.5 | 1 | 1.5 | 1.5 | 2.5 | Total |
| | | | | | | | Volume (ml) |
| 0.018 | 0.029 | 0.337 | 0.419 | 0.509 | 0.278 | 0.060 | A₅₆₂ |
| 3.6 | 6 | 68 | 84 | 102 | 56 | 12 | Stock conc. |
| | | | | | | | (μg/ml) |
| 9 | 15 | 238 | 84 | 153 | 84 | 30 | Total Weight |
| | | | | | | | (μg) |

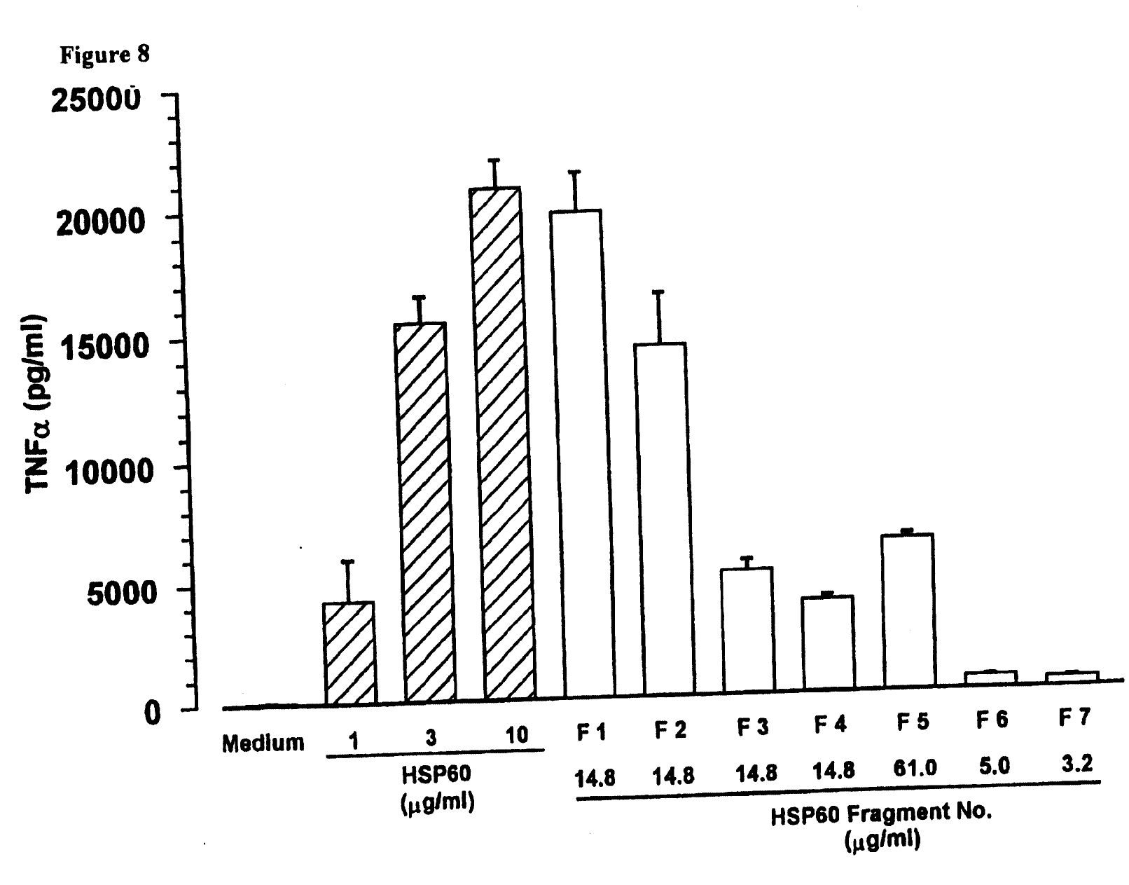

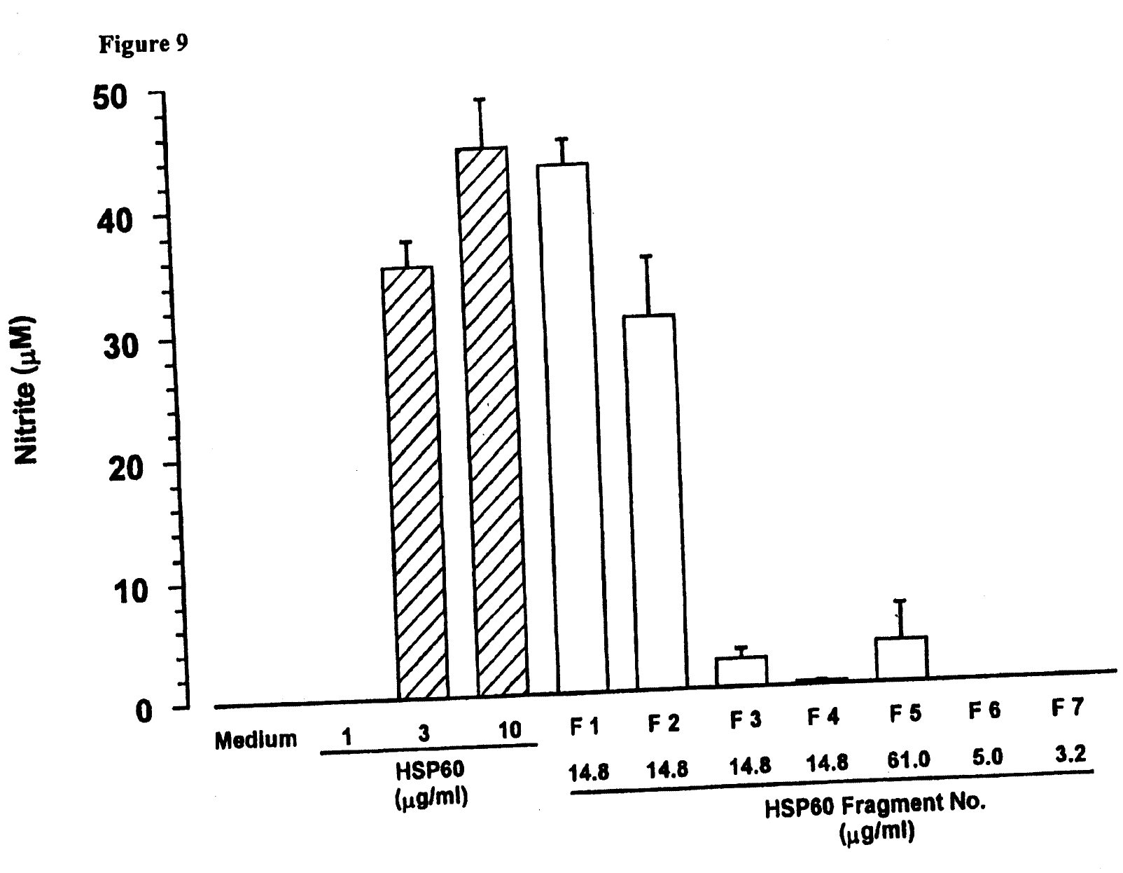

All fractions were concentrated by Centricon YM-3 (2 ml volume) and then by microcon YM-3 (0.5 ml volume), both with cutoff of 3000 dalton, to a concentration of about 1 mg/ml. The fractions were tested for their ability to induce the production of TNFα and NO as described above with the exception that J774 cells were used (Chen et al., J. Immunol. 162 (1999), 3212). The concentrations of the tryptic fragments added to the J774 macrophage cultures are indicated in FIGS. 8 and 9, as well as the results of these experiments. Fractions 1 and 2 were found to be active.

2. Separation of Fraction 2 of Trypsin-Degraded-Hsp60 by Reverse Phase HPLC

Fifty μl of fraction 2 were separated by reverse phase HPLC on a C-4 column (Vydac214TP54, Protein RP-C4, 250×4.6 mm, 5μ) using a gradient of 0.1% TFA in water and 0.1% TFA in acetonitryl for 75 minutes at 40° C.

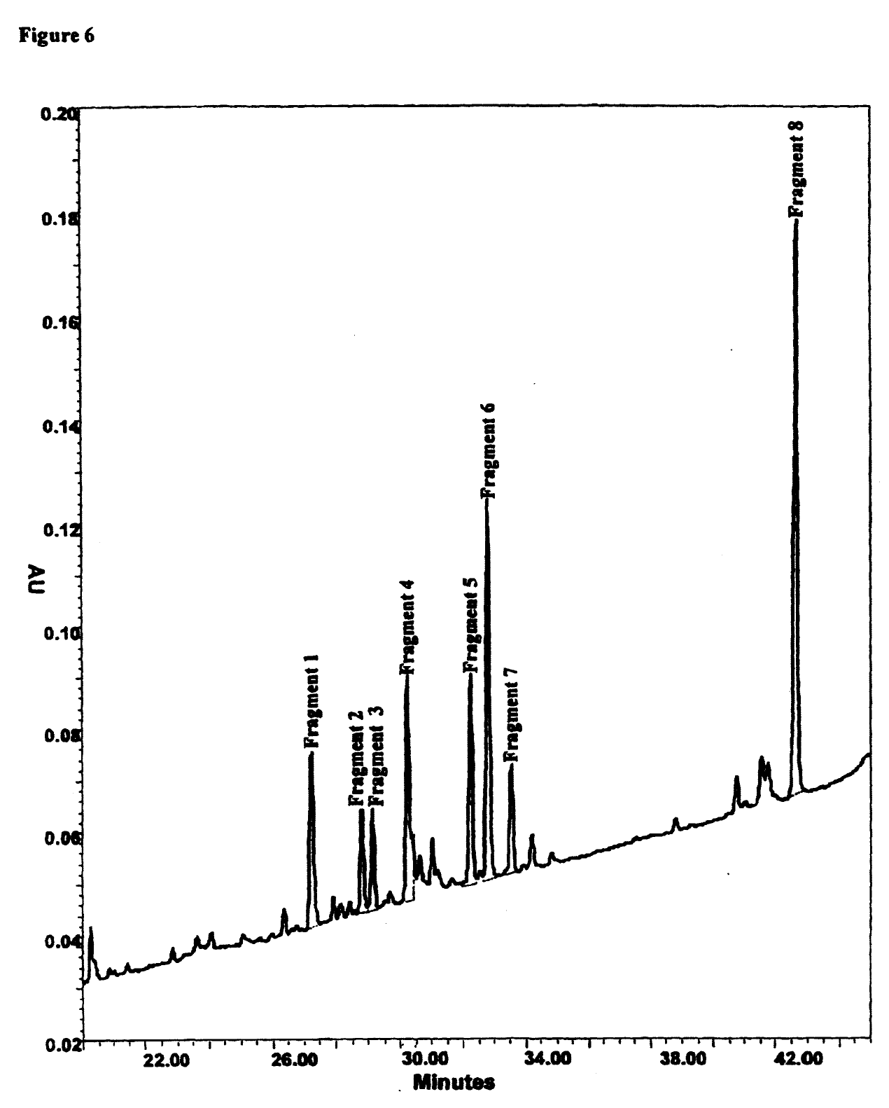

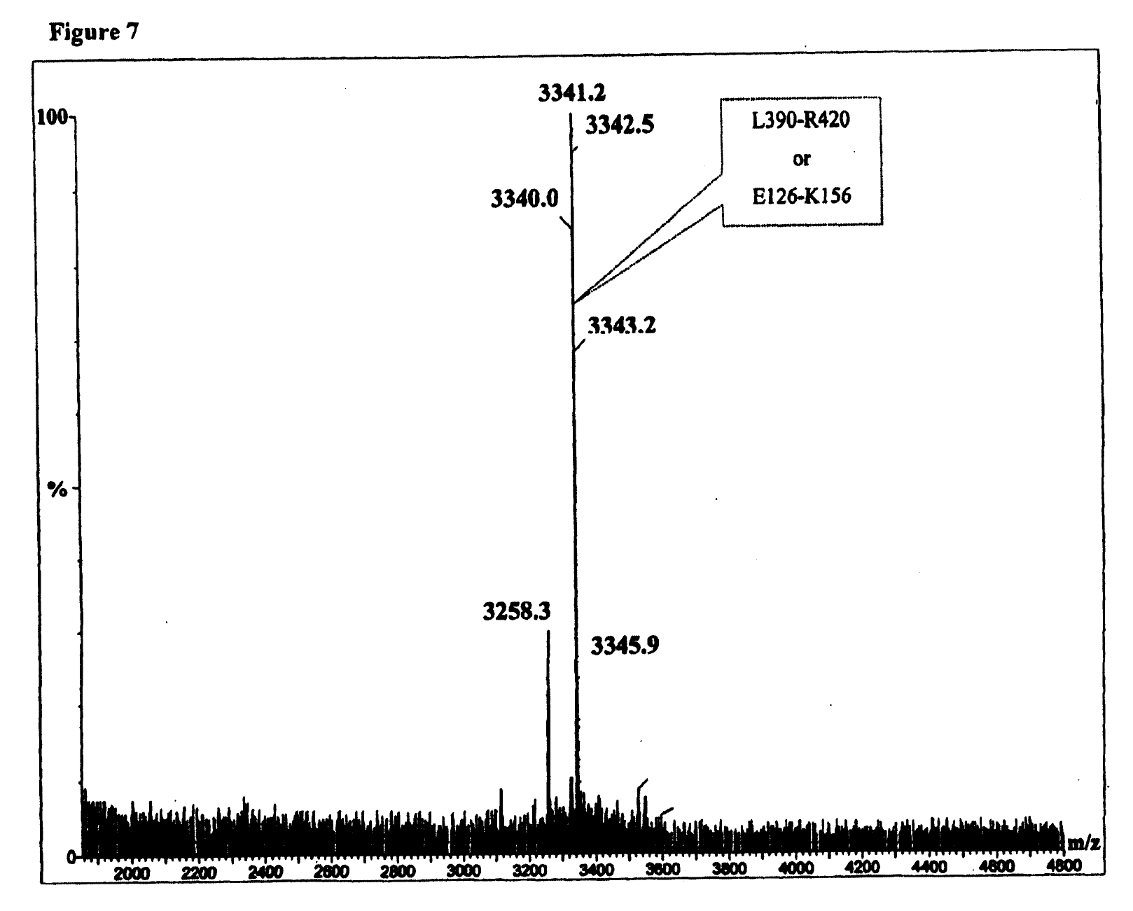

The results (absorbance at 215.0 nm) are summarized in FIG. 6. The eight main peaks obtained were collected and analyzed by Edman-degradation for their N-terminal sequence, and by MALDI-TOF Mass Spectrometry for their mass. In each of the eight fractions one or more masses were found. The found masses were matched to the possible tryptic fragments of hsp60. An example is given in FIG. 7 which shows the MS analysis results of peak number 3.

In total there are five regions which are represented by the identified fragments:

1. One mass which is within one of E₁₂₆-K₁₅₆ or L₃₉₀-R₄₂₀.

2. The region K₁₅₇-K₁₉₂ with four exact masses and one possible.

3. The region G₂₂₂-K₂₄₉ with one exact mass.

4. The region K₂₆₉-R₂₉₀ with one exact mass.

5. The region K₃₁₀-K₃₄₄ with two exact masses.

3. Structural Analysis of Identified Fragments

The fragments identified by Mass Spectroscopy of the active fraction of the tryptic digest are (note that fragments within the same group show a large degree of overlap):

1) Glu₁₂₆-Lys₁₅₆

2) Lys₁₅₇-Lys₁₉₁

Gln₁₅₈-Lys₁₉₂

Gln₁₅₈-Lys₁₉₁

Pro₁₆₁-Lys₁₉₁

Pro₁₆₁-Lys₁₉₂

3) Gly₂₂₂-Lys₂₄₉

4) Lys₂₆₉-Arg₂₉₀

5) Lys₃₁₀-Lys₃₄₄

Asp₃₁₅-Lys₃₄₄

6) Leu₃₉₀-Val₄₂₁

In order to analyze these results several approaches were pursued concomitantly:

a) A 3-dimensional model of human hsp60 was built by homology from the crystallographically known structure of its bacterial homologue-GROEL. This allowed to determine, for each of the fragments that was found within the active fraction of the tryptic digest, which are expected to be exposed to solvent within the framework of the entire hsp60 protein.

b) The sequence of spaetzle, the natural ligand of the toll receptor, was aligned with that of hsp60, and several fragments of varying degree of homology were identified (see Scheme I). This allowed to determine which of the hsp60 fragments found within the active fraction of the digest can potentially bind to the recognition sites of the toll receptor.

c) The secondary structure of the spaetzle protein was predicted. This enabled, for each of the fragments in the active fraction of the tryptic digest that showed homology to a fragment of spaetzle, a structural comparison.

Analysis of the above listed six regions showed:

Region 1 shows a significant degree of homology with two fragments of spaetzle (this region and the alignment are marked as Box 1 in Scheme I (legend: * identical residues, : very similar residues, . similar residues). The arrow in the box denotes that the fragment continues on the next line).

Region 2 does not show sequence homology to spaetzle, yet spans two solvent exposed helices within hsp60, along with two loops, one preceding the first helix and the other between the two. Both the loops and the helices are solvent exposed and are thus potential recognition sites on hsp60. Since spaetzle is predicted to be a four-helix bundle, a structural homology between Region 2 and spaetzle that enables recognition by the same receptor is possible, despite lack of obvious sequence homology within this region.

Region 3 shows significant homology with a single fragment of spaetzle (marked as Box 2 in the Scheme I). This region is a highly flexible, surface exposed loop in the GROEL crystal structure, and hence in the hsp60 model. The secondary structure of spaetzle in the region homologous to Region 3 is also predicted to be a loop. Thus all of the analyses suggest this fragment to be a likely candidate for recognition by the toll receptor.

Region 4 shows significant homology with a fragment of spaetzle (marked as Box 3 in Scheme I). It is only partially exposed in the hsp60 model. Structural comparison between this region and the corresponding region in spaetzle is not straightforward, since a large part of this fragment shows poor electron density in the GROEL crystal.

Region 5 spans a large fragment of hsp60 that shows no sequence homology with spaetzle tailed by a short fragment that shows homology (Box 4 in Scheme I). The C-terminal part of this fragment (which includes the Box 4 sequence and an additional 9 residues upstream, are surface exposed).

Region 6 shows homology with two fragments of spaetzle (Box 5 in Scheme I; the arrow marks the continuation of this region to the next line). The entire fragment consists of a surface exposed helix, followed by a short P-strand, a short, partially exposed loop and an additional helix. The spaetzle fragment homologous to the first helix is predicted to be a loop, yet the fragment homologous to the C-terminal helix is predicted to be a helix also.

An additional region of hsp60 spanning Lys₃₅₉ to Asp₃₉₉ shows a significant degree of homology to a fragment of spaetzle, and is solvent exposed. Despite that fact that a fragment corresponding to this region was not observed in the active fraction of the tryptic digest, it will be investigated whether this region is responsible for activity.

Since each of these analyses involves some degree of uncertainty, a requirement to conform with all of them is not realistic. Thus each of the above seven regions (the six from the Mass Spectroscopy analysis and the additional mentioned region) are investigated by a Multiple-Parallel Synthesis of arrays of overlapping backbone cyclized analogs of Peptide Loop(s) in the size ranges of 5-10 residues, designed to optimally mimic the structure of the fragment within the hsp60 model. The arrays were focused on structured backbone cyclized analogs, having sequences identical or close to the native, yet entrapping these sequences in varying constrained conformations. Once an active peptide analog is identified, arrays of backbone cyclized analogs having the same overall conformation as the active one yet with variations in the sequence will be designed. These latter analogs may improve the affinity towards the toll receptor.

### Example 3

### Tryptic Digest of Hsp60 and Screening of Fragments

A larger scale batch of hsp60 tryptic digest fragments was produced using a similar procedure to the one described in example no. 2.

Three major fractions 1, 2, and 3, were obtained by size exclusion FPLC separation. Each fraction was then sub-fractionated by reverse-phase HPLC. The peaks were collected separately and lyophilized.

The major fractions and the separated peaks were tested for stimulatory and inhibitory effect on TNFα secretion in J774A cells.

Results: stimulation activity was detected in fractions 1 and 2.

### Example 4

### Synthesis and Screening of 20-Mer Overlapping Peptides

Thirty-six overlapping peptides of 20-residue length with overlap of 10 residues (and shift of ten residues between adjacent peptides) were synthesized spanning the region of residues 111-490 of the human hsp60.

The peptides were tested for inhibition of hsp60-induced TNF secretion.

The preliminary results are summarized in Table No. 2:

TABLE 2
Activity of 20-mer peptide fragments of hsp60

| Inhibitory | Weakly inhibitory | Not inhibitory |
|---|---|---|
| 111-130 | | |
| | | 121-140 |
| | | 131-150 |
| 141-160 | | |
| | | 151-170 |
| | | 161-180 |
| | | 171-190 |
| | 181-200 | |
| | 191-210 | |
| | | 201-220 |
| 211-230 | | |
| | | 221-240 |
| | 231-250 | |
| | | 241-260 |
| 251-270 | | |
| | | 261-280 |
| | | 271-290 |
| | | 281-300 |
| | | 291-310 |
| | | 301-320 |
| | | 311-330 |
| | 321-340 | |
| | 331-350 | |
| | 341-360 | |
| | | 351-370 |
| | | 361-380 |
| | | 371-390 |
| 381-400 | | |
| 391-410 | | |
| 401-420 | | |
| | | 421-440 |
| 431-450 | | |
| 441-460 | | |
| 451-470 | | |
| 461-480 | | |
| 471-490 | | |

### Example 5

### Design of Overlapping 10-Mer Peptide Fragments of Human Hsp60

In order to find an active short fragment of hsp60, 182 peptide fragments of human hsp60 were designed based on integration of all data available as indicated in Table No. 3. This includes:

1) The results (activity+MS analysis) of the hsp60 first fragmentation (tryptic digestion) study.

2) A 3D model of hsp60; specifically an analysis of the surface accessibility of each of the above fragments.

3) Homology to spaetzle (both sequence and structural, based on a model of spaetzle).

4) The activity results of the 20-mer overlapping peptides spanning residues 111-490 of the human hsp60.

TABLE 3
Parameters for designing the 10-mer peptides

| Region | Spaetzle | Spaetzle | | 20-mer | |
|---|---|---|---|---|---|
| Residue range | sequence | structure | Surface | peptides | Number of |
| in hsp60 | homology | homology | accessibility | activity | peptides |
| 1) 126-160 | + | − | + | +++ | 26 |
| 2) 140-192 | − | + | + | +++ | 32 |
| 3) 220-250 | + | + | + | ++ | 22 |
| 4) 269-290 | + | + | −/+ | ++ | 13 |
| 5) 310-344 | −/+ | −/+ | −/+ | + | 26 |
| 6) 390-421 | + | +/− | + | n.t. | 23 |
| 7) 350-400 | + | + | + | n.t. | 40 |

Notes:
1) Regions 1-6 are consistent with the fragmentation studies. Region 7 did not turn up in the fragmentation studies, yet its homology with spaetzle and its apparent surface exposure suggested that it is a region worth screening.
2) Structural homology to spaetzle is based on a first approximation model.
3) All sequences refer to the sequence of human hsp60 (SWISSPORT database accession No. P10809 and Scheme I).
4) n.t. = not tested.
5) There are 182 peptides altogether
6) Ten peptides were repeated twice in the plate (to give 192 for two 96-well plates): Five peptides from first region of the 20-mer active peptide. Five peptides from central portion of region 7 above.

Plates 1+2 contain 192 deca-peptides: 58 peptides span residues 126-192 of human hsp60, 22 span residues 220-250, 13 span residues 269-290, 26 peptides span residues 310-344 and 63 peptides span residues 350-421, all with shift of one amino acid. (10 additional peptides are repeats of 5 peptides from region 2 and 5 from region 7).

### Example 6

### Synthesis of the 192 Peptides of 10-Mer

The peptides were synthesized in multiple parallel synthesis (MPS) format using an automatic peptide synthesizer (ACT 396 of Advanced ChemTech) and heating device (Lab Tech 4 of Advanced ChemTech). Two plates of 96 peptides each were synthesized. The peptides were synthesized by solid-phase method using the procedure recommended by the synthesizer's producer, briefly:

Synthesis scale: 6 μmol peptide per well.

Resin: Rink amide

Chemistry: Fmoc chemistry, deprotection with 25% piperidine in NMP.

Couplings: with 5 equivalents of HBTU/HOBT as a coupling reagent and 10 equivalents of DIEA as a base.

Cleavage: with a solution of 92.5% TFA, 2.5% H₂O, 2.5% TIS, 2.5% EDT.

Partial purification: performed by passing the cleaved peptides through Sep-Pak (C-18) plate columns.

The peptides were tested by mass spectral analysis. Expected mass of most peptides was found.

### Example 7

### Screening of the 10-Mer Peptides for Inhibition of Hsp60

The crude peptides were re-suspended in 500 μl water and tested at a dilution of 1:10, for inhibition of hsp60 induced TNFα and Nitric oxide (NO) secretion in J774 cells (as described in example no. 1 of the application). Selected peptides were tested (in an equivalent assay) also for inhibition of CpG induced TNFα secretion. CpG is a non-specific immunostimulatory oligonucleotide motif with the sequence: 5′-TCCATAACGTTGCAAACGTTCTG-3′ (Krieg AM. CpG DNA: a novel immunomodulator Trends Microbiol 1999; 7:64-5). CpG stimulates J774 cells to produce TNFα in a mechanism different to that used by hsp60 and thus serves as a negative control for non-selective inhibition of TNF secretion (which is not mediated through specific inhibition of hsp60). Preferred peptides of the present invention inhibit hsp60 induced but not CpG induced-TNFα secretion.

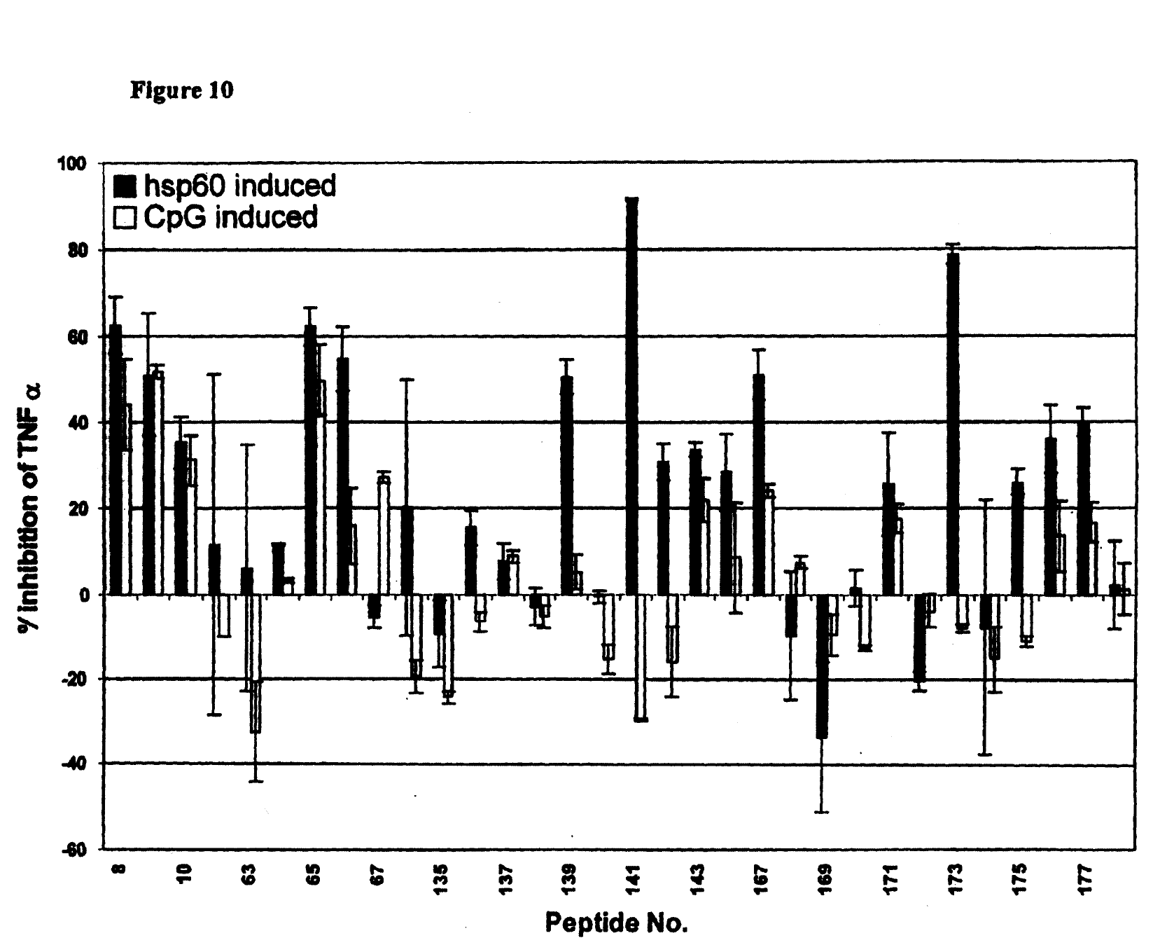

The activity results of selected peptides are summarized in FIG. 10. The peptide sequences are summarized in table No. 4. The peptides in this chart were selected for re-screening based on the activity found in screening all the 192 peptides synthesized. The results are presented as percent inhibition of TNF-secretion in response to induction with hsp60 or CpG, and are the average of two repeats.

TABLE 4
Amino acid sequence of selected 10-mer peptides

| | Residue No. | | | | | | | | | |
|---|---|---|---|---|---|---|---|---|---|---|
| Peptide No. | 1 | 2 | 3 | 4 | 5 | 6 | 7 | 8 | 9 | 10 |
| 8 | Lys¹³³ | Gly | Ala | Asn | Pro | Val | Glu | Ile | Arg | Arg |
| 9 | Gly | Ala | Asn | Pro | Val | Glu | Ile | Arg | Arg | Gly |
| 10 | Ala | Asn | Pro | Val | Glu | Ile | Arg | Arg | Gly | Val |
| 11 | Asn¹³⁶ | Pro | Val | Glu | Ile | Arg | Arg | Gly | Val | Met |
| 63 | Ile²²⁴ | Ser | Pro | Tyr | Phe | Ile | Asn | Thr | Ser | Lys |
| 64 | Ser | Pro | Tyr | Phe | Ile | Asn | Thr | Ser | Lys | Gly |
| 65 | Pro | Tyr | Phe | Ile | Asn | Thr | Ser | Lys | Gly | Gln |
| 66 | Tyr²²⁷ | Phe | Ile | Asn | Thr | Ser | Lys | Gly | Gln | Lys |
| 67 | Phe²²⁸ | Ile | Asn | Thr | Ser | Lys | Gly | Gln | Lys | Cys |
| 134 | Lys³⁶⁴ | Ala | Gln | Ile | Glu | Lys | Arg | Ile | Gln | Glu |
| 135 | Ala | Gln | Ile | Glu | Lys | Arg | Ile | Gln | Glu | Ile |
| 136 | Gln | Ile | Glu | Lys | Arg | Ile | Gln | Glu | Ile | Ile |
| 137 | Ile | Glu | Lys | Arg | Ile | Gln | Glu | Ile | Ile | Glu |
| 138 | Glu | Lys | Arg | Ile | Gln | Glu | Ile | Ile | Glu | Gln |
| 139 | Lys³⁶⁹ | Arg | Ile | Gln | Glu | Ile | Ile | Glu | Gln | Leu |
| 140 | Arg | Ile | Gln | Glu | Ile | Ile | Glu | Gln | Leu | Asp |
| 141 | Gln³⁷² | Gln | Glu | Ile | Ile | Glu | Gln | Leu | Asp | Val |
| 142 | Gln³⁷² | Glu | Ile | Ile | Glu | Gln | Leu | Asp | Val | Thr |
| 143 | Glu | Ile | Ile | Glu | Gln | Leu | Asp | Val | Thr | Thr |
| 144 | Ile³⁷⁴ | Ile | Glu | Gln | Leu | Asp | Val | Thr | Thr | Ser |
| 167 | Leu³⁹⁷ | Ser | Asp | Gly | Val | Ala | Val | Leu | Lys | Val |
| 168 | Ser | Asp | Gly | Val | Ala | Val | Leu | Lys | Ser | Gly |
| 169 | Asp | Gly | Val | Ala | Val | Leu | Lys | Val | Gly | Gly |
| 170 | Gly | Val | Ala | Val | Leu | Lys | Val | Gly | Gly | Thr |
| 171 | Val | Ala | Val | Leu | Lys | Val | Gly | Gly | Thr | Ser |
| 172 | Ala | Val | Leu | Lys | Val | Gly | Gly | Thr | Ser | Asp |
| 173 | Val⁴⁰³ | Leu | Lys | Val | Gly | Gly | Thr | Ser | Asp | Val |
| 174 | Leu | Lys | Val | Gly | Gly | Thr | Ser | Asp | Val | Glu |
| 175 | Lys⁴⁰⁵ | Val | Gly | Gly | Thr | Ser | Asp | Val | Glu | Val |
| 176 | Val | Gly | Gly | Thr | Ser | Asp | Val | Glu | Val | Asn |
| 177 | Gly | Gly | Thr | Ser | Asp | Val | Glu | Val | Asn | Glu |
| 178 | Gly⁴⁰⁸ | Thr | Ser | Asp | Val | Glu | Val | Asn | Glu | Lys |

The most active and specific analogs from this set of peptides are:

Peptide No. 66 spanning residues 227-236 of hsp60

Peptide No. 134 spanning residues 364-373 of hsp60

Peptide No. 139 spanning residues 369-378 of hsp60

Peptide No. 141 spanning residues 371-380 of hsp60

Peptide No. 142 spanning residues 372-381 of hsp60

Peptide No. 173 spanning residues 403-412 of hsp60

Peptide No. 175 spanning residues 405-414 of hsp60

### Example 8

### Design of Additional Peptides from Residues 364-381 of Human Hsp60

A second generation of overlapping peptides was designed based on the screening of the 10-mer peptides. This set aim to study the region spanning residues 364-381 of the human hsp60 sequence (KAQIEKRIQEIIEQLDVT), since within this region several peptides demonstrated specific inhibition of TNF secretion.

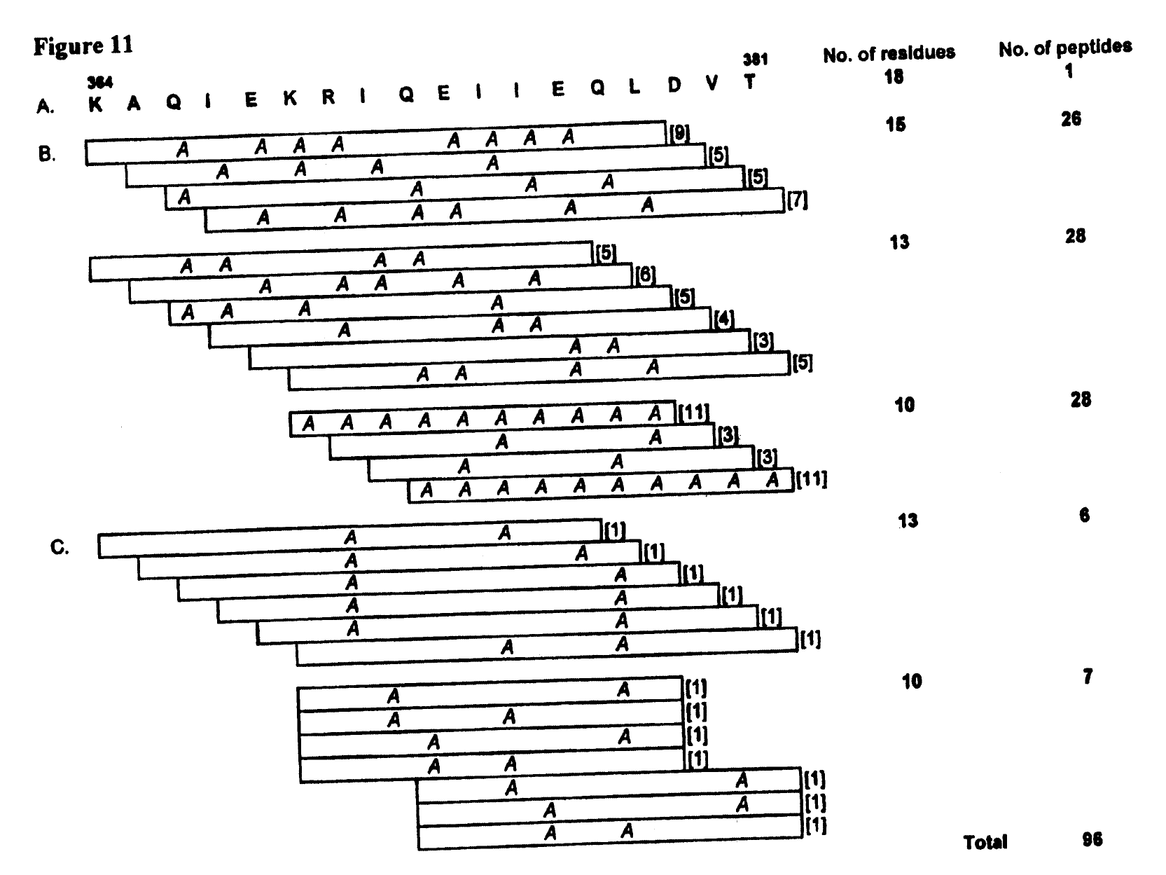

FIG. 11 describes the 96 peptides which include:

A. An 18-mer peptide spanning the entire helix that is found in this region of the hsp60 model;

B. Three sets of overlapping peptides of 10, 13 and 15 residues including the wild-type peptides and peptides with one amino acid replaced with Alanine (Alanine-scan, A represent the position replaced with Alanine);

C. Two sets of overlapping peptides of 10 and 13 residues including the wild-type peptides and peptides with two amino acids replaced with Alanine (double position Alanine-scan);

### Example 9

### Design of Backbone Cyclized Peptides

A second generation of peptides derived from region 7 was also designed, since within this region hsp60-inhibiting peptides were found (peptides 173 and 175 of table 4). The plate contains backbone cyclized peptides based on the sequence of residues 408-416 of hsp60. The peptides (table 5), are 9-residues long with four types of cyclizations, bridging between positions 408-414, 409-416, 408-416, and 409-414. Several types of building units (labeled bold in table 5), have been utilized as bridging groups in order to form different bridge sizes and types. The Asn residue at position 415 has been replaced with Gln or another residue due to synthetic considerations. In addition (table 6), linear peptides based on residues 403-412 of hsp60 were designed.

TABLE 5

| 408 | 409 | 410 | 411 | 412 | 413 | 414 | 415 | 416 |
|---|---|---|---|---|---|---|---|---|
| GlyC1 | Thr | Ser | Asp | Val | Glu | AlaN2 | Gln | Glu |
| PheC1 | Thr | Ser | Asp | Val | Glu | AlaN2 | Gln | Glu |
| GlyC2 | Thr | Ser | Asp | Val | Glu | AlaN2 | Gln | Glu |
| TrpC2 | Thr | Ser | Asp | Val | Glu | AlaN2 | Gln | Glu |
| GlyC2 | Thr | Ser | Asp | Val | Glu | AlaN3 | Gln | Glu |
| GlyC3 | Thr | Ser | Asp | Val | Glu | AlaN2 | Gln | Glu |
| GlyC3 | Thr | Ser | Asp | Val | Glu | AlaN3 | Gln | Glu |
| LeuC4 | Thr | Ser | Asp | Val | Glu | AlaN2 | Gln | Glu |
| PheC4 | Thr | Ser | Asp | Val | Glu | AlaN2 | Gln | Glu |
| LysC4 | Thr | Ser | Asp | Val | Glu | AlaN2 | Gln | Glu |
| LeuC4 | Thr | Ser | Asp | Val | Glu | AlaN3 | Gln | Glu |
| PheC4 | Thr | Ser | Asp | Val | Glu | AlaN3 | Gln | Glu |
| LysC4 | Thr | Ser | Asp | Val | Glu | AlaN3 | Gln | Glu |
| LeuC5 | Thr | Ser | Asp | Val | Glu | AlaN2 | Gln | Glu |
| TrpC5 | Thr | Ser | Asp | Val | Glu | AlaN3 | Gln | Glu |
| GlyC2 | Thr | Ser | Asp | Val | Glu | Val | Gln | LeuN4 |
| TrpC2 | Thr | Ser | Asp | Val | Glu | Val | Ser | LeuN4 |
| GlyN2 | Thr | Ser | Asp | Val | Glu | Val | Gln | AlaC3 |
| LeuN2 | Thr | Ser | Asp | Val | Glu | Val | Thr | PheC3 |
| SerN2 | Thr | Ser | Asp | Val | Glu | Val | Gln | AlaC3 |
| GlyC3 | Thr | Ser | Asp | Val | Glu | Val | Gln | HISN3 |
| PheC3 | Thr | Ser | Asp | Val | Glu | Val | Gln | LysN3 |
| IleC3 | Thr | Ser | Asp | Val | Glu | Val | Gln | PheN3 |
| GlyC3 | Thr | Ser | Asp | Val | Glu | Val | Gln | PheN3 |
| GlyC3 | Thr | Ser | Asp | Val | Glu | Val | Ala | LeuN4 |
| TrpC3 | Thr | Ser | Asp | Val | Glu | Val | Gln | GlyN6 |
| LeuC4 | Thr | Ser | Asp | Val | Glu | Val | Gln | GluN2 |
| PheC4 | Thr | Ser | Asp | Val | Glu | Val | Gln | GluN2 |
| LysC4 | Thr | Ser | Asp | Val | Glu | Val | Gln | GluN2 |
| LeuC4 | Thr | Ser | Asp | Val | Glu | Val | Gln | LysN2 |
| PheC4 | Thr | Ser | Asp | Val | Glu | Val | Gln | PheN2 |
| GlyN2 | Thr | Ser | Asp | Val | Glu | Val | Gln | TyrC4 |
| TrpN2 | Thr | Ser | Asp | Val | Glu | Val | Gln | AlaC4 |
| ThrN2 | Thr | Ser | Asp | Val | Glu | Val | Thr | LeuC4 |
| SerN2 | Thr | Ser | Asp | Val | Glu | Val | Gln | MetC4 |
| LeuN2 | Thr | Ser | Asp | Val | Glu | Val | 2Abu | PheC4 |
| LeuC4 | Thr | Ser | Asp | Val | Glu | Val | Gln | PheN3 |
| PheC4 | Thr | Ser | Asp | Val | Glu | Val | Gln | TrpN3 |
| LysC4 | Thr | Ser | Asp | Val | Glu | Val | Leu | LeuN3 |
| LeuC4 | Thr | Ser | Asp | Val | Glu | Val | Thr | TrpN3 |
| PheC4 | Thr | Ser | Asp | Val | Glu | Val | Gln | AlaN3 |
| LeuC4 | Thr | Ser | Asp | Val | Glu | Val | Gln | LysN3 |
| PheC4 | Thr | Ser | Asp | Val | Glu | Val | Gln | LysN3 |
| LysC4 | Thr | Ser | Asp | Val | Glu | Val | Gln | HISN3 |
| AlaC5 | Thr | Ser | Asp | Val | Glu | Val | Gln | GluN2 |
| LeuC5 | Thr | Ser | Asp | Val | Glu | Val | Gln | GluN2 |
| TrpC5 | Thr | Ser | Asp | Val | Glu | Val | Gln | GluN2 |
| AlaC5 | Thr | Ser | Asp | Val | Glu | Val | Gln | LysN2 |
| LeuC5 | Thr | Ser | Asp | Val | Glu | Val | Gln | AlaN2 |
| TrpC5 | Thr | Ser | Asp | Val | Glu | Val | Ala | TrpN2 |
| LysN2 | Thr | Ser | Asp | Val | Glu | Val | Ser | LeuC5 |
| TrpN2 | Thr | Ser | Asp | Val | Glu | Val | Gln | TrpC5 |
| Gly | ThrN2 | Ser | Asn | Val | Glu | AlaC2 | Gln | Glu |
| Gly | ThrN2 | Ser | Gln | Val | Glu | AlaC2 | Gln | Glu |
| Gly | ThrN2 | Ser | Glu | Val | Glu | AlaC2 | Gln | Glu |
| Gly | ThrN2 | Ser | Lys | Val | Glu | AlaC2 | Gln | Glu |
| Gly | ThrN2 | Ser | Tyr | Val | Glu | AlaC2 | Gln | Lys |
| Gly | SerN2 | Ser | Asn | Val | Tyr | AlaC2 | Gln | Glu |
| Gly | SerN2 | Ser | Gln | Val | Glu | AlaC2 | Gln | Glu |
| Gly | ThrN2 | 2Abu | Asn | Val | Glu | AlaC2 | Gln | Glu |
| Gly | ThrN2 | Ser | Gln | Val | Glu | AlaC2 | Gln | Asp |
| Gly | ThrN2 | Ser | Glu | Val | Glu | AlaC2 | Gln | Tyr |
| Gly | ThrN2 | Ser | Lys | Val | Glu | AlaC2 | Gln | Phe |
| Gly | AlaC3 | Ser | Asp | Val | Glu | AlaN2 | Gln | Glu |
| Gly | LeuC3 | Ser | Asp | Val | Glu | AlaN2 | Gln | Glu |
| Gly | PheC3 | Ser | Asp | Val | Glu | AlaN2 | Gln | Glu |
| Gly | AlaC3 | Ser | Asp | Val | Glu | AlaN3 | Gln | Glu |
| Gly | LeuC3 | Ser | Asp | Val | Glu | AlaN3 | Gln | Glu |
| Gly | PheC3 | Ser | Asp | Val | Glu | AlaN3 | Gln | Glu |
| Gly | ThrN2 | Ser | Asp | Val | Glu | AlaC2 | Gln | Glu |
| Gly | ThrN2 | Thr | Asp | Val | Glu | AlaC2 | Gln | Asp |
| Gly | ThrN2 | Val | Asp | Leu | Glu | AlaC2 | Gln | Lys |
| Gly | ThrN2 | 2Abu | Asp | 2Abu | Glu | AlaC2 | Gln | Glu |
| Gly | ThrN2 | Phe | Asp | Ile | Glu | AlaC2 | Gln | Glu |
| Gly | SerN2 | Leu | Asp | Ala | Glu | AlaC2 | Gln | Glu |
| Gly | SerN2 | Thr | Asp | Leu | Glu | AlaC2 | Gln | Glu |
| Gly | ThrN2 | Ser | Asp | Val | Glu | Val | Gln | TyrC4 |
| Gly | ThrN2 | Ser | Asp | Val | Glu | Val | Tyr | LeuC4 |
| Gly | LeuC3 | Ser | Asp | Val | Glu | Val | Gln | GluN2 |
| Gly | PheC3 | Ser | Asp | Val | Glu | Val | Gln | GluN2 |
| Gly | ValC3 | Ser | Asp | Val | Glu | Val | Gln | GluN2 |
| Gly | LeuC3 | Ser | Asp | Val | Glu | Val | Gln | PheN3 |
| Gly | PheC3 | Ser | Asp | Val | Glu | Val | Thr | TyrN3 |
| Gly | ValC3 | 2Abu | Asp | Val | Glu | Val | Gln | PheN3 |
| Gly | LeuC3 | Ser | Asp | Val | Glu | Val | Gln | TyrN3 |
| Gly | PheC3 | Ser | Asp | Val | Glu | Val | Gln | TyrN3 |
| Gly | ValC3 | Ser | Asp | Val | Glu | Val | Gln | PheN3 |
| Gly | LeuC3 | Ser | Asp | Val | Glu | Val | 2Abu | TyrN3 |
| Gly | PheC3 | Ser | Asp | Val | Glu | Val | Gln | PheN3 |
| Gly | ValC3 | Ser | Asp | Val | Glu | Val | Gln | TyrN3 |
| Gly | Thr | Ser | Asp | Val | Glu | Val | Gln | Glu |

TABLE 6

| | | | | | | | | | | | | |
|---|---|---|---|---|---|---|---|---|---|---|---|---|
| | | | Val | Leu | Lys | Val | Gly | Gly | Thr | Ser | Asp | Val |
| Val | Leu | Lys | Val | Gly | Gly | Thr | Ser | Asp | Val | Glu | Val | Asn |
| Ala | Val | Leu | Lys | Val | Gly | Gly | Thr | Ser | Asp | Val | Glu | Val |
| Val | Ala | Val | Leu | Lys | Val | Gly | Gly | Thr | Ser | Asp | Val | Val |
| Gly | Val | Ala | Val | Leu | Lys | Val | Gly | Gly | Thr | Ser | Asp | Val |

While the present invention has been described for certain preferred embodiments and examples it will be appreciated by the skilled artisan that many variations and modifications may be performed to optimize the activities of the peptides and analogs of the invention. The examples are to be construed as non-limitative and serve only for illustrative purposes of the principles disclosed according to the present invention, the scope of which is defined by the claims which follow.

What is claimed is:

1. A variant, derivative, analog or peptide fragment of hsp60, the variant, derivative, analog, or peptide fragment having the ability to act as an antagonist of hsp60 characterized by its ability to reduce or prevent the induction of a pro-inflammatory immune response of cells of the innate immune system by hsp60, by inhibiting the binding of hsp60 to a Toll like receptor.

2. The antagonist of hsp60 according to claim 1 comprising a peptide sequence of five to thirty amino acids.

3. The antagonist of hsp60 according to claim 1 comprising a peptide sequence of five to twenty-five amino acids.

4. The antagonist of hsp60 according to claim 1 comprising a peptide sequence of seven to eighteen amino acids.

5. The antagonist of hsp60 according to claim 1 having a peptide sequence derived from a fragment of hsp60 selected from the group consisting of:
(a) Residues 126-160 of human hsp60;
(b) Residues 140-192 of human hsp60;
(c) Residues 220-250 of human hsp60;
(d) Residues 269-290 of human hsp60;
(e) Residues 310-344 of human hsp60;
(f) Residues 390-421 of human hsp60; and
(g) Residues 350-400 of human hsp60.

6. The antagonist according to claim 5 wherein the fragment of hsp 60 is selected from the group consisting of:
(a) Residues 126-156 of human hsp60;
(b) Residues 157-191 of human hsp60;
(c) Residues 158-192 of human hsp60;
(d) Residues 158-191 of human hsp60;
(e) Residues 161-191 of human hsp60;
(f) Residues 161-192 of human hsp60;
(g) Residues 222-249 of human hsp60;
(h) Residues 310-344 of human hsp60; and
(i) Residues 315-344 of human hsp60.

7. The antagonist of hsp60 according to claim 6 having a sequence selected from the group consisting of:
(a) Tyr-Phe-Ile-Asn-Thr-Ser-Lys-Gly-Gln-Lys;
(b) Lys-Ala-Gln-Ile-Glu-Lys-Arg-Ile-Gln-Glu;
(c) Lys-Arg-Ile-Gln-Glu-Ile-Ile-Glu-Gln-Leu;
(d) Ile-Gln-Glu-Ile-Ile-Glu-Gln-Leu-Asp-Val;
(e) Gln-Glu-Ile-Ile-Glu-Gln-Leu-Asp-Val-Thr;
(f) Val-Leu-Lys-Val-Gly-Gly-Thr-Ser-Asp-Val;
(g) Lys-Val-Gly-Gly-Thr-Ser-Asp-Val-Glu-Val;
(h) Lys-Ala-Gln-Ile-Glu-Lys-Arg-Ile-Gln-Glu-Ile-Ile-Ala-Gln-Leu;
(i) Lys-Arg-Ala-Gln-Glu-Ile-Ile-Glu-Ala-Leu;
(j) Val-Leu-Lys-Val-Gly-Gly-Thr-Ser-Asp-Val-Glu-Val-Asn;
(k) Ala-Val-Leu-Lys-Val-Gly-Gly-Thr-Ser-Asp-Val-Glu-Val;
(l) Val-Ala-Val-Leu-Lys-Val-Gly-Gly-Thr-Ser-Asp-Val-Val; and
(m) Gly-Val-Ala-Val-Leu-Lys-Val-Gly-Gly-Thr-Ser-Asp-Val.

8. A backbone cyclized peptide analog of a fragment of hsp60, the analog incorporating at least one building unit, the building unit containing at least one nitrogen atom of the peptide backbone connected to a bridging group comprising an amide, thioether, thioester or disulfide, wherein at least one building unit is connected via the bridging group to form a cyclic structure with a moiety selected from the group consisting of a second building unit, the side chain of an amino acid residue of the sequence or the terminal amino acid residue, the analog having the ability to act as an antagonist of hsp60 characterized by its ability to reduce or prevent the induction of a pro-inflammatory immune response of cells of the innate immune system by hsp60.

9. The backbone cyclized peptide analog of claim 8 wherein the fragment of hsp60 has a having a peptide sequence derived from a fragment of hsp60 selected from the group consisting of:
(a) Residues 126-160 of human hsp60;
(b) Residues 140-192 of human hsp60;
(c) Residues 220-250 of human hsp60;
(d) Residues 269-290 of human hsp60;
(e) Residues 310-344 of human hsp60;
(f) Residues 390-421 of human hsp60; and
(g) Residues 350-400 of human hsp60.

10. A backbone cyclized peptide analog of residues 408-416 of hsp60.

11. The backbone cyclized peptide analog according to claim 10 wherein the cyclic structure is formed by bridging two building units located at positions selected from the group consisting of:
(a) Residues 408 and 414 of hsp60;
(b) Residues 409 and 416 of hsp60;
(c) Residues 408 and 416 of hsp60; and
(d) Residues 409 and 414 of hsp60.

12. A method for identifying fragments of hsp60 having the ability to elicit a pro-inflammatory immune response comprising the steps of
(a) contacting cells of the innate immune system carrying on their surface an intact Toll-like receptor complex with a fragment of hsp60; and
(b) determining the amount of a pro-inflammatory mediator or effector, produced by the cells;
wherein an increase in the production of the pro-inflammatory mediator or effector, is indicative for the ability of the fragment of hsp60 to elicit a pro-inflammatory response by the innate immune system.

13. The method according to claim 12 wherein step (b) comprises determining the amount of TNFα and/or NO produced by the cells;
wherein an increase in the production of TNFα and/or NO is indicative for the ability of the fragment of hsp60 to elicit a pro-inflammatory response by the innate immune system.

14. A method for identifying, screening and/or characterizing compounds which can act as antagonists of hsp60 characterized in that they can reduce or prevent the induction of a pro-inflammatory response by hsp60, wherein this method comprises the steps of:
(a) contacting cells of the innate immune system which carry on their surface a Toll-like receptor complex with hsp60 or a fragment thereof having the ability to induce a pro-inflammatory response in the presence and in the absence of the compound to be tested; and
(b) determining whether the presence of the compound has an effect on the production of a pro-inflammatory mediator or effector, of the cells,
wherein a reduction or prevention of the production of the pro-inflammatory mediator or effector, is indicative of the ability of the compound to act as an antagonist of hsp60.

15. The method according to claim 14 wherein step (b) comprises
determining the amount of TNFα and/or NO produced by the cells;
wherein a reduction or prevention of the production TNFα and/or NO is indicative of the ability of the compound to act as an antagonist of hsp60.

16. A pharmaceutical composition comprising as an active ingredient an antagonist of hsp60 according to claim 1.

17. A method of preparing a pharmaceutical composition for the treatment of an inflammatory disease or disorder, which comprises:
identifying a fragment of hsp60 according to claim 12 wherein the fragment has the ability to elicit a pro-inflammatory immune response; and
selecting the fragment to be the biologically active ingredient of the composition.

18. The method of claims 17, wherein the fragment is provided in the composition along with a pharmaceutically acceptable carrier or diluent.

19. The method of claim 17 wherein the inflammatory disease is selected from: an autoimmune disease, diabetes, multiple sclerosis, rheumatoid arthritis, myasthenia gravis, systemic lupus erythematosis chronic inflammation, atherosclerosis, inflammatory bowel disease, reactive arthritis, chronic infections, graft rejection, gingivitis, gastritis, ulcer, thrombosis, allergy and acute infections.

20. A method of preparing a pharmaceutical composition for the treatment of an inflammatory disease or disorder, which comprises:
providing an antagonist of hsp60 in the form of a variant, derivative, analog or peptide fragment according to claim 1; and
incorporating the antagonist in the composition as the biologically active ingredient.

21. The method of claim 20 wherein the inflammatory disease is selected from: an autoimmune disease, diabetes, multiple sclerosis, rheumatoid arthritis, myasthenia gravis, systemic lupus erythematosis chronic inflammation, atherosclerosis, inflammatory bowel disease, reactive arthritis, chronic infections, graft rejection, gingivitis, gastritis, ulcer, thrombosis, allergy and acute infections.

22. A method of treating an inflammatory disease or disorder in a patient in need of such treatment, which comprises administering an antagonist of hsp60 in the form of a variant, derivative, analog or peptide fragment according to claim 1 to the patient where the antagonist is present in an amount that is therapeutically effective to treat the disease or disorder.

23. The method of claim 22 wherein the inflammatory disease is selected from: an autoimmune disease, diabetes, multiple sclerosis, rheumatoid arthritis, myasthenia gravis, systemic lupus erythematosis chronic inflammation, atherosclerosis, inflammatory bowel disease, reactive arthritis, chronic infections, graft rejection, gingivitis, gastritis, ulcer, thrombosis, allergy and acute infections.

24. A method of treating an inflammatory disease or disorder in a patient in need of such treatment, which comprises administering the pharmaceutical composition of claim 16 to the patient where the active ingredient is present in an amount that is therapeutically effective to treat the disease or disorder.

25. The method of claim 24 wherein the inflammatory disease is selected from: an autoimmune disease, diabetes, multiple sclerosis, rheumatoid arthritis, myasthenia gravis, systemic lupus erythematosis chronic inflammation, atherosclerosis, inflammatory bowel disease, reactive arthritis, chronic infections, graft rejection, gingivitis, gastritis, ulcer, thrombosis, allergy and acute infections.


SEQUENCE LISTING (125 sequences)

SEQ ID NO: 1 - protein, 573 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 2 - protein, 35 aa, Homo sapiens
EGFEKISKGANPVEIRRGVMLAVDAVIAELKKQSK

SEQ ID NO: 3 - protein, 31 aa, Homo sapiens
EGFEKISKGANPVEIRRGVMLAVDAVIAELK

SEQ ID NO: 4 - protein, 53 aa, Homo sapiens
IRRGVMLAVDAVIAELKKQSKPVTTPEEIAQVATISANGDKEIGNIISDAMKK

SEQ ID NO: 5 - protein, 35 aa, Homo sapiens
KQSKPVTTPEEIAQVATISANGDKEIGNIISDAMK

SEQ ID NO: 6 - protein, 35 aa, Homo sapiens
QSKPVTTPEEIAQVATISANGDKEIGNIISDAMKK

SEQ ID NO: 7 - protein, 34 aa, Homo sapiens
QSKPVTTPEEIAQVATISANGDKEIGNIISDAMK

SEQ ID NO: 8 - protein, 31 aa, Homo sapiens
PVTTPEEIAQVATISANGDKEIGNIISDAMK

SEQ ID NO: 9 - protein, 32 aa, Homo sapiens
PVTTPEEIAQVATISANGDKEIGNIISDAMKK

SEQ ID NO: 10 - protein, 31 aa, Homo sapiens
DRGYISPYFINTSKGQKCEFQDAYVLLSEKK

SEQ ID NO: 11 - protein, 29 aa, Homo sapiens
RGYISPYFINTSKGQKCEFQDAYVLLSEK

SEQ ID NO: 12 - protein, 22 aa, Homo sapiens
KPLVIIAEDVDGEALSTLVLNR

SEQ ID NO: 13 - protein, 35 aa, Homo sapiens
KNQLKDMAIATGGAVFGEEGLTLNLEDVQPHDLGK

SEQ ID NO: 14 - protein, 34 aa, Homo sapiens
NQLKDMAIATGGAVFGEEGLTLNLEDVQPHDLGK

SEQ ID NO: 15 - protein, 32 aa, Homo sapiens
LNERLAKLSDGVAVLKVGGTSDVEVNEKKDRV

SEQ ID NO: 16 - protein, 41 aa, Homo sapiens
VTKDDAMLLKGKGDKAQIEKRIQEIIEQLDVTTSEYEKEKL

SEQ ID NO: 17 - protein, 10 aa, Homo sapiens
YFINTSKGQK

SEQ ID NO: 18 - protein, 10 aa, Homo sapiens
KAQIEKRIQE

SEQ ID NO: 19 - protein, 10 aa, Homo sapiens
KRIQEIIEQL

SEQ ID NO: 20 - protein, 10 aa, Homo sapiens
IQEIIEQLDV

SEQ ID NO: 21 - protein, 10 aa, Homo sapiens
VLKVGGTSDV

SEQ ID NO: 22 - protein, 10 aa, Homo sapiens
KVGGTSDVEV

SEQ ID NO: 23 - protein, 18 aa, Homo sapiens
KAQIEKRIQEIIEQLDVT

SEQ ID NO: 24 - protein, 15 aa, Homo sapiens
KAQIEKRIQEIIAQL

SEQ ID NO: 25 - protein, 10 aa, Homo sapiens
KRAQEIIEAL

SEQ ID NO: 26 - protein, 13 aa, Homo sapiens
VLKVGGTSDVEVN

SEQ ID NO: 27 - protein, 13 aa, Homo sapiens
AVLKVGGTSDVEV

SEQ ID NO: 28 - protein, 13 aa, Homo sapiens
VAVLKVGGTSDVV

SEQ ID NO: 29 - protein, 13 aa, Homo sapiens
GVAVLKVGGTSDV

SEQ ID NO: 30 - protein, 10 aa, Homo sapiens
VLKVGGTSDV

SEQ ID NO: 31 - protein, 13 aa, Homo sapiens
VLKVGGTSDVEVN

SEQ ID NO: 32 - protein, 13 aa, Homo sapiens
AVLKVGGTSDVEV

SEQ ID NO: 33 - protein, 13 aa, Homo sapiens
VAVLKVGGTSDVV

SEQ ID NO: 34 - protein, 13 aa, Homo sapiens
GVAVLKVGGTSDV

SEQ ID NO: 35 - protein, 9 aa, Artificial Sequence
  Modified peptides of Homo sapiens HSP60
XTSDVEXNE

SEQ ID NO: 36 - protein, 9 aa, Artificial Sequence
  Modified peptides of Homo sapiens HSP60
XTSDVEXNE

SEQ ID NO: 37 - protein, 9 aa, Artificial Sequence
  Modified peptides of Homo sapiens HSP60
XTSDVEXNE

SEQ ID NO: 38 - protein, 9 aa, Artificial Sequence
  Modified peptides of Homo sapiens HSP60
XTSDVEXNE

SEQ ID NO: 39 - protein, 9 aa, Artificial Sequence
  Modified peptides of Homo sapiens HSP60
XTSDVEXNE

SEQ ID NO: 40 - protein, 9 aa, Artificial Sequence
  Modified peptides of Homo sapiens HSP60
XTSDVEXNE

SEQ ID NO: 41 - protein, 9 aa, Artificial Sequence
  Modified peptides of Homo sapiens HSP60
XTSDVEXNE

SEQ ID NO: 42 - protein, 9 aa, Artificial Sequence
  Modified peptides of Homo sapiens HSP60
XTSDVEXNE

SEQ ID NO: 43 - protein, 9 aa, Artificial Sequence
  Modified peptides of Homo sapiens HSP60
XTSDVEXNE

SEQ ID NO: 44 - protein, 9 aa, Artificial Sequence
  Modified peptides of Homo sapiens HSP60
XTSDVEXNE

SEQ ID NO: 45 - protein, 9 aa, Artificial Sequence
  Modified peptides of Homo sapiens HSP60
XTSDVEXNE

SEQ ID NO: 46 - protein, 9 aa, Artificial Sequence
  Modified peptides of Homo sapiens HSP60
XTSDVEXNE

SEQ ID NO: 47 - protein, 9 aa, Artificial Sequence
  Modified peptides of Homo sapiens HSP60
XTSDVEXNE

SEQ ID NO: 48 - protein, 9 aa, Artificial Sequence
  Modified peptides of Homo sapiens HSP60
XTSDVEXNE

SEQ ID NO: 49 - protein, 9 aa, Artificial Sequence
  Modified peptides of Homo sapiens HSP60
XTSDVEXNE

SEQ ID NO: 50 - protein, 9 aa, Artificial Sequence
  Modified peptides of Homo sapiens HSP60
XTSDVEVNX

SEQ ID NO: 51 - protein, 9 aa, Artificial Sequence
  Modified peptides of Homo sapiens HSP60
XTSDVEVSX

SEQ ID NO: 52 - protein, 9 aa, Artificial Sequence
  Modified peptides of Homo sapiens HSP60
XTSDVEVNX

SEQ ID NO: 53 - protein, 9 aa, Artificial Sequence
  Modified peptides of Homo sapiens HSP60
XTSDVEVTX

SEQ ID NO: 54 - protein, 9 aa, Artificial Sequence
  Modified peptides of Homo sapiens HSP60
XTSDVEVNX

SEQ ID NO: 55 - protein, 9 aa, Artificial Sequence
  Modified peptides of Homo sapiens HSP60
XTSDVEVNX

SEQ ID NO: 56 - protein, 9 aa, Artificial Sequence
  Modified peptides of Homo sapiens HSP60
XTSDVEVNX

SEQ ID NO: 57 - protein, 9 aa, Artificial Sequence
  Modified peptides of Homo sapiens HSP60
XTSDVEVNX

SEQ ID NO: 58 - protein, 9 aa, Artificial Sequence
  Modified peptides of Homo sapiens HSP60
XTSDVEVNX

SEQ ID NO: 59 - protein, 9 aa, Artificial Sequence
  Modified peptides of Homo sapiens HSP60
XTSDVEVAX

SEQ ID NO: 60 - protein, 9 aa, Artificial Sequence
  Modified peptides of Homo sapiens HSP60
XTSDVEVNX

SEQ ID NO: 61 - protein, 9 aa, Artificial Sequence
  Modified peptides of Homo sapiens HSP60
XTSDVEVNX

SEQ ID NO: 62 - protein, 9 aa, Artificial Sequence
  Modified peptides of Homo sapiens HSP60
XTSDVEVNX

SEQ ID NO: 63 - protein, 9 aa, Artificial Sequence
  Modified peptides of Homo sapiens HSP60
XTSDVEVNX

SEQ ID NO: 64 - protein, 9 aa, Artificial Sequence
  Modified peptides of Homo sapiens HSP60
XTSDVEVNX

SEQ ID NO: 65 - protein, 9 aa, Artificial Sequence
  Modified peptides of Homo sapiens HSP60
XTSDVEVNX

SEQ ID NO: 66 - protein, 9 aa, Artificial Sequence
  Modified peptides of Homo sapiens HSP60
XTSDVEVNX

SEQ ID NO: 67 - protein, 9 aa, Artificial Sequence
  Modified peptides of Homo sapiens HSP60
XTSDVEVNX

SEQ ID NO: 68 - protein, 9 aa, Artificial Sequence
  Modified peptides of Homo sapiens HSP60
XTSDVEVTX

SEQ ID NO: 69 - protein, 9 aa, Artificial Sequence
  Modified peptides of Homo sapiens HSP60
XTSDVEVNX

SEQ ID NO: 70 - protein, 9 aa, Artificial Sequence
  Modified peptides of Homo sapiens HSP60
XTSDVEVEX

SEQ ID NO: 71 - protein, 9 aa, Artificial Sequence
  Modified peptides of Homo sapiens HSP60
XTSDVEVNX

SEQ ID NO: 72 - protein, 9 aa, Artificial Sequence
  Modified peptides of Homo sapiens HSP60
XTSDVEVNX

SEQ ID NO: 73 - protein, 9 aa, Artificial Sequence
  Modified peptides of Homo sapiens HSP60
XTSDVEVLX

SEQ ID NO: 74 - protein, 9 aa, Artificial Sequence
  Modified peptides of Homo sapiens HSP60
XTSDVEVTX

SEQ ID NO: 75 - protein, 9 aa, Artificial Sequence
  Modified peptides of Homo sapiens HSP60
XTSDVEVNX

SEQ ID NO: 76 - protein, 9 aa, Artificial Sequence
  Modified peptides of Homo sapiens HSP60
XTSDVEVNX

SEQ ID NO: 77 - protein, 9 aa, Artificial Sequence
  Modified peptides of Homo sapiens HSP60
XTSDVEVNX

SEQ ID NO: 78 - protein, 9 aa, Artificial Sequence
  Modified peptides of Homo sapiens HSP60
XTSDVEVNX

SEQ ID NO: 79 - protein, 9 aa, Artificial Sequence
  Modified peptides of Homo sapiens HSP60
XTSDVEVNX

SEQ ID NO: 80 - protein, 9 aa, Artificial Sequence
  Modified peptides of Homo sapiens HSP60
XTSDVEVNX

SEQ ID NO: 81 - protein, 9 aa, Artificial Sequence
  Modified peptides of Homo sapiens HSP60
XTSDVEVNX

SEQ ID NO: 82 - protein, 9 aa, Artificial Sequence
  Modified peptides of Homo sapiens HSP60
XTSDVEVNX

SEQ ID NO: 83 - protein, 9 aa, Artificial Sequence
  Modified peptides of Homo sapiens HSP60
XTSDVEVNX

SEQ ID NO: 84 - protein, 9 aa, Artificial Sequence
  Modified peptides of Homo sapiens HSP60
XTSDVEVAX

SEQ ID NO: 85 - protein, 9 aa, Artificial Sequence
  Modified peptides of Homo sapiens HSP60
XTSDVEVSX

SEQ ID NO: 86 - protein, 9 aa, Artificial Sequence
  Modified peptides of Homo sapiens HSP60
XTSDVEVNX

SEQ ID NO: 87 - protein, 9 aa, Artificial Sequence
  Modified peptides of Homo sapiens HSP60
GXSNVEXNE

SEQ ID NO: 88 - protein, 9 aa, Artificial Sequence
  Modified peptides of Homo sapiens HSP60
GXSNVEXNE

SEQ ID NO: 89 - protein, 9 aa, Artificial Sequence
  Modified peptides of Homo sapiens HSP60
GXSEVEXNE

SEQ ID NO: 90 - protein, 9 aa, Artificial Sequence
  Modified peptides of Homo sapiens HSP60
GXSKVEXNE

SEQ ID NO: 91 - protein, 9 aa, Artificial Sequence
  Modified peptides of Homo sapiens HSP60
GXSYVEXNK

SEQ ID NO: 92 - protein, 9 aa, Artificial Sequence
  Modified peptides of Homo sapiens HSP60
GXSNVYXNE

SEQ ID NO: 93 - protein, 9 aa, Artificial Sequence
  Modified peptides of Homo sapiens HSP60
GXSNVEXNE

SEQ ID NO: 94 - protein, 9 aa, Artificial Sequence
  Modified peptides of Homo sapiens HSP60
GXENVEXNE

SEQ ID NO: 95 - protein, 9 aa, Artificial Sequence
  Modified peptides of Homo sapiens HSP60
GXSNVEXND

SEQ ID NO: 96 - protein, 9 aa, Artificial Sequence
  Modified peptides of Homo sapiens HSP60
GXSEVEXNY

SEQ ID NO: 97 - protein, 9 aa, Artificial Sequence
  Modified peptides of Homo sapiens HSP60
GXSKVEXNF

SEQ ID NO: 98 - protein, 9 aa, Artificial Sequence
  Modified peptides of Homo sapiens HSP60
GXSDVEXNE

SEQ ID NO: 99 - protein, 9 aa, Artificial Sequence
  Modified peptides of Homo sapiens HSP60
GXSDVEXNE

SEQ ID NO: 100 - protein, 9 aa, Artificial Sequence
  Modified peptides of Homo sapiens HSP60
GXSDVEXNE

SEQ ID NO: 101 - protein, 9 aa, Artificial Sequence
  Modified peptides of Homo sapiens HSP60
GXSDVEXNE

SEQ ID NO: 102 - protein, 9 aa, Artificial Sequence
  Modified peptides of Homo sapiens HSP60
GXSDVEXNE

SEQ ID NO: 103 - protein, 9 aa, Artificial Sequence
  Modified peptides of Homo sapiens HSP60
GXSDVEXNE

SEQ ID NO: 104 - protein, 9 aa, Artificial Sequence
  Modified peptides of Homo sapiens HSP60
GXSDVEXNE

SEQ ID NO: 105 - protein, 9 aa, Artificial Sequence
  Modified peptides of Homo sapiens HSP60
GXTDVEXNE

SEQ ID NO: 106 - protein, 9 aa, Artificial Sequence
  Modified peptides of Homo sapiens HSP60
GXVDLEXNE

SEQ ID NO: 107 - protein, 9 aa, Artificial Sequence
  Modified peptides of Homo sapiens HSP60
GXEDEEXNE

SEQ ID NO: 108 - protein, 9 aa, Artificial Sequence
  Modified peptides of Homo sapiens HSP60
GXFDIEXNE

SEQ ID NO: 109 - protein, 9 aa, Artificial Sequence
  Modified peptides of Homo sapiens HSP60
GXLDAEXNE

SEQ ID NO: 110 - protein, 9 aa, Artificial Sequence
  Modified peptides of Homo sapiens HSP60
GXTDLEXNE

SEQ ID NO: 111 - protein, 9 aa, Artificial Sequence
  Modified peptides of Homo sapiens HSP60
GXSDVEVNX

SEQ ID NO: 112 - protein, 9 aa, Artificial Sequence
  Modified peptides of Homo sapiens HSP60
GXSDVEVYX

SEQ ID NO: 113 - protein, 9 aa, Artificial Sequence
  Modified peptides of Homo sapiens HSP60
GXSDVEVNX

SEQ ID NO: 114 - protein, 9 aa, Artificial Sequence
  Modified peptides of Homo sapiens HSP60
GXSDVEVNX

SEQ ID NO: 115 - protein, 9 aa, Artificial Sequence
  Modified peptides of Homo sapiens HSP60
GXSDVEVNX

SEQ ID NO: 116 - protein, 9 aa, Artificial Sequence
  Modified peptides of Homo sapiens HSP60
GXSDVEVNX

SEQ ID NO: 117 - protein, 9 aa, Artificial Sequence
  Modified peptides of Homo sapiens HSP60
GXSDVEVTX

SEQ ID NO: 118 - protein, 9 aa, Artificial Sequence
  Modified peptides of Homo sapiens HSP60
GXEDVEVNX

SEQ ID NO: 119 - protein, 9 aa, Artificial Sequence
  Modified peptides of Homo sapiens HSP60
GXSDVEVNX

SEQ ID NO: 120 - protein, 9 aa, Artificial Sequence
  Modified peptides of Homo sapiens HSP60
GXSDVEVNX

SEQ ID NO: 121 - protein, 9 aa, Artificial Sequence
  Modified peptides of Homo sapiens HSP60
GXSDVEVNX

SEQ ID NO: 122 - protein, 9 aa, Artificial Sequence
  Modified peptides of Homo sapiens HSP60
GXSDVEVEX

SEQ ID NO: 123 - protein, 9 aa, Artificial Sequence
  Modified peptides of Homo sapiens HSP60
GXSDVEVNX

SEQ ID NO: 124 - protein, 9 aa, Artificial Sequence
  Modified peptides of Homo sapiens HSP60
GXSDVEVNX

SEQ ID NO: 125 - protein, 9 aa, Homo sapiens
GTSDVEVNE